(19) United States
(12) Patent Application Publication
(10) Pub. No.: US 2024/0215826 A1
(43) Pub. Date: Jul. 4, 2024

(54) DEVICES AND METHODS FOR IN VIVO TISSUE EVALUATION

(71) Applicant: Board of Regents, The University of Texas System, Austin, TX (US)

(72) Inventors: Panayiotis Shiakolas, Arlington, TX (US); Samson Adejokun, Arlington, TX (US); Shashank Kumat, Arlington, TX (US)

(21) Appl. No.: 18/403,416

(22) Filed: Jan. 3, 2024

Related U.S. Application Data
(60) Provisional application No. 63/436,784, filed on Jan. 3, 2023.

(51) Int. Cl. A61B 5/00; A61B 90/00

(52) U.S. Cl. CPC A61B 5/0053; A61B 5/6846; A61B 90/06; A61B 2090/064; A61B 2560/0406; A61B 2562/0261

(57) ABSTRACT

According to some embodiments, a device for tissue evaluation comprises a sensor housing, the sensor housing comprising a sensor head and a sensor body, where the sensor head is configured to engage with target tissue for evaluation, and a sensing element disposed within the sensor housing and configured to measure a force or reaction force of the target tissue as the device is pressed against the target tissue.

### CROSS-REFERENCE TO RELATED APPLICATIONS

This application claims priority pursuant to 35 U.S.C. § 119(e) to U.S. Provisional Application Ser. No. 63/436,784, filed on Jan. 3, 2023, which is hereby incorporated by reference in its entirety.

### FIELD

The technology described herein generally relates to devices and method for tissue evaluation, more specifically to micro force sensors for in vivo tissue evaluation and the use thereof.

### BACKGROUND

Urinary bladder cancer is considered as a leading cause of patient mortality among cancers, with 83,730 estimated new cases in 2021, corresponding to 4.4% of all new cancer cases in the US alone. Tests performed for bladder cancer diagnosis include physical exam and health history, endoscopy, urinalysis, urine cytology, cystoscopy, intravenous pyelogram (IVP) and biopsy. Diagnostic tests such as physical exam, internal exam urine cytology and cystoscopy cannot be used to quantitatively identify localized in vivo tissue viscoelastic properties. Some of the current technologies employed to interact with tissue in confined spaces include endoscopy, cystoscopy, prolapse assessment, biopsy, and analysis of the exterior bladder wall movement.

Urinary incontinence (UI) is another medical condition affecting 33 million Americans classified as either stress urinary incontinence or urge urinary incontinence. Ex vivo tensile testing demonstrated that the tissue stiffness index helps to identify the severity of pelvic floor disorder and UI.

Studies showed that a tumorous surface exhibits a higher stiffness compared to healthier surrounding tissues. As such, tumor mechanics significantly differ from that of normal tissue. Hence, identifying localized viscoelastic properties of tissues can be advantageous in assessing the healthiness of organs.

Instrumentation used in clinical settings limits a physician's ability to deliver consistent care. Therefore, there is a need for diagnostic tools capable of accessing confined spaces in the body to interact with the tissue at the local level and then use interaction results to evaluate quantifiable tissue properties for medical diagnosis and care. For example, the quantification of vaginal tissue alterations could aid in detecting women at risk of pelvic organ prolapse (POP) at an early stage.

One study showed that pelvic organs can undergo a reaction force of approximately 1.2 N with an indentation depth ranging from 8-10 mm. Monitoring of tissue viscoelastic properties over a time period could lead to the development of better treatments and outcomes for women suffering from POP. Therefore, evaluation of localized tissue properties will be essential for disease prevention and/or detection. Being able to monitor the applied force and acquire reaction force information could result in improved diagnostics. Therefore, there has been interest in the development of micro-force sensors that could be attached at the end of diagnostic instruments to interact with the tissue.

Accordingly, the technology described herein provides improvements over conventional tissue evaluation, specifically in vivo tissue evaluation through the implementation of methods and devices described herein for example through the implementation of miniature or micro force sensors. Accordingly, tissue healthiness could be assessed by evaluating its viscoelastic properties through localized contact reaction force measurements to obtain quantitative time history information. To evaluate these properties for hard to reach and confined areas of the human body, miniature force sensors with size constraints and appropriate load capabilities are needed. The quantitative characterization of soft tissue viscoelastic properties can aid in disease prognosis and diagnosis. Existing technologies present challenges to measuring localized in-vivo tissue relaxation data while meeting load and geometric constraints.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are generally directed towards a sensor (or micro force sensor or miniature force sensor) for in vivo tissue evaluation and/or measurement that is further fabricated by a three-dimensional printing technique.

According to some embodiments, a device for in vivo tissue evaluation is provided. The device can comprise a sensor housing and a sensing element. The sensor housing can comprise a sensor head configured to engage with target tissue and a sensor body. The sensing element can be disposed within the sensor housing. Additionally, the device can be configured to measure a force or a reaction force of the target tissue when the sensor and/or sensor head is pushed or otherwise pressed against the target tissue.

According to some further embodiments a method for measuring a force or reaction force of tissue (e.g. in vivo target tissue) is provided. The method can comprise implanting a sensor device having a sensor head such that the sensor head is substantially perpendicular to and in contact with a target tissue. The sensor device can be pressed or pushed against the target tissue causing the sensor head to move relative to a sensor body of the device. The sensor and/or sensor head can receive a force or applied force from the target tissue against the sensor head and cause the sensing element to deform in response to the displacement of the sensor head. The sensor and/or sensing element or one or more gauges integrated or attached to the sensing element can then generate one or more signals corresponding to a force or reaction force of the target tissue.

Additional objects, advantages, and novel features of the technology will be set forth in part in the description which follows, and in part will become apparent to those skilled in the art upon examination of the following, or can be learned by practice of the invention.

### BRIEF DESCRIPTION OF THE DRAWINGS

Aspects of the technology presented herein are described in detail below with reference to the accompanying drawing figures, wherein:

FIG. 1a shows a schematic illustrating an example sensor or sensor device in accordance with some aspects of the technology described herein;

FIG. 1b shows a schematic illustrating an example sensor or sensor device in accordance with some aspects of the technology described herein;

FIG. 2 illustrates an example sensor element finite element FE analysis model, in accordance with some aspects of the technology described herein;

FIG. 3 illustrates a schematic of an example data acquisition system and calibration system for a sensor, in accordance with some aspects of the technology described herein;

FIG. 4 illustrates an example testbed for the characterization of a sensor, in accordance with some aspects of the technology described herein;

FIG. 5 illustrates an example theoretical strain evaluation of a sensor or sensing element, in accordance with some aspects of the technology described herein;

FIG. 6A illustrates a side view orientation of an example sensing element, in accordance with some aspects of the technology described herein;

FIG. 6B illustrates a front view orientation of an example sensing element, in accordance with some aspects of the technology described herein;

FIG. 7 illustrates example fabrication of a sensor head component fabricated in different orientations, in accordance with some aspects of the technology described herein;

FIG. 8 illustrates an example fabricated sensor base or body, in accordance with some aspects of the technology described herein;

FIG. 9A illustrates a top view of an example cured sensor base or body component, in accordance with some aspects of the technology described herein;

FIG. 9B illustrates a bottom view of an example cured sensor head component, in accordance with some aspects of the technology described herein;

FIG. 9C illustrates an example of a prototyped sensor, in accordance with some aspects of the technology described herein;

FIG. 10 illustrates an evaluated strain as a function of applied load for an example sensor, in accordance with some aspects of the technology described herein;

FIG. 11 illustrates an in vivo tissue response from a sensor, in accordance with some aspects of the technology described herein;

FIG. 12 shows example specifications and characteristics of a sensor, in accordance with some aspects of the technology described herein;

FIGS. 13a and 13b illustrate schematic models of a foil strain gauge and an example sensor base component; in accordance with some aspects of the technology described herein;

FIG. 14 illustrates aspects of an example sensor; in accordance with some aspects of the technology described herein;

FIG. 15 illustrates a schematic of an example sensor; in accordance with some aspects of the technology described herein;

FIG. 16 illustrates an example finite element-based model for a sensor, in accordance with some aspects of the technology described herein;

FIG. 17 illustrates an example finite element-based model due to a maximum applied load on a sensor; in accordance with some aspects of the technology described herein;

FIG. 18 illustrates evaluations of example design points for a sensor, in accordance with some aspects of the technology described herein;

FIG. 19 shows the effect of bend radius and bend angle for a sensing element at an applied load in light of objective function of the sensing element, in accordance with some aspects of the technology described herein;

FIG. 20 shows an example calibration platform, in accordance with some aspects of the technology described herein;

FIG. 21 shows sensitivity responses as a function of force for a load cell, in accordance with some aspects of the technology described herein;

FIG. 22 shows load-cell and post-processed sensor data, in accordance with some aspects of the technology described herein;

FIG. 23 shows evaluated force time history for a sensor, in accordance with some aspects of the technology described herein;

FIG. 24 shows sensor repeatability band, in accordance with some aspects of the technology described herein;

FIG. 25 shows hysteresis responses from an example calibrated sensor, in accordance with some aspects of the technology described herein;

FIG. 26A illustrates an example sensor, in accordance with some aspects of the technology described herein; and

FIG. 26B illustrates an example sensor, in accordance with some aspects of the technology described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

According to embodiments of the present technology, a force sensor (or micro force sensor, or miniature force sensor, and/or uniaxial force sensor) for in vivo tissue evaluation and/or measurement is provided. In some instances, a strain gauge based or integrated sensor comprising a head component, a body and/or base component, and a sensing element is provided. The sensor components can in some instances meet dimensional constraints (diameter≤3.5 mm), safety factor (≥3) and performance specifications (for example maximum applied load, resolution, sensitivity, and accuracy). The sensing element can in some instances be manufactured through a machining process. Three-dimensional printing techniques, such as inverted vat photopolymerization for example, can be used to fabricate complex components, for example on a Form3 printer.

Described herein are aspects of the design, component fabrication using three-dimensional printing technology, integration, characterization, and analysis of in vivo collected measurements. In some instances, a force sensor according to the present technology can be implemented for measurement, evaluation, and/or assessment of tissue viscoelastic properties. Further, a force sensor can be used to obtain tissue quantitative data to assess tissue healthiness, for example for medical care, and over extended time periods.

According to various embodiments, three dimensional (3D) printed components can exhibit good dimensional accuracy (maximum deviation of 183 μm). The assembled sensor can exhibit linear behavior (regression coefficient of R2=0.999) and can meet performance specifications of, for example, 3.4 safety factor, 1.2 N load capacity, 18 mN resolution, and 3.13% accuracy. The in vivo experimentally obtained relaxation data were analyzed using the Voigt model yielding a viscoelastic coefficient τ=12.38 sec and a curve-fit regression coefficient of R2=0.992.

A micro-force sensor with diameter less than 3 mm and a total length of 15 mm capable of sensing an axial load in the range 0-4 N with a resolution of 14 mN was previously reported. Conventional manufacturing processes including laser machining were used for prototyping a Nitinol alloy based force sensor and for prototyping subsequent design revisions aiming to simplify sensor design. The design suffered from incorporating necessary complex features due to limitations of conventional manufacturing processes. A triaxial force sensor with a diameter of 4 mm was previously designed and was placed at the tip of a catheter for measuring the interaction force between tissues. This sensor had an accuracy and resolution of 2.7 mN and 0.6 mN respectively in the axial direction and capable of withstanding an axial load of 0.8 N. Components of the force sensor were fabricated using 3D printing technology due to the presence of complex features. These other sensors are based upon the working principle of fiber optics. Fiber brag grating (FBG) sensing technology can be used as a strain measurement sensor by measuring the change in period of the wavelength of the emitted light source. The use of FBG limits the ability of the sensor holding equipment to attain large bending angles)(˜180° and small bending radius in confined spaces such as the bladder. In addition, FBG instrumentation is bulky and costly which could further limit widespread use of FBG based diagnostic devices. The limitations of FBG based sensors could be overcome by strain gauge based force sensors. A strain gauge based force sensor is a well-developed and mature technology and its working principle relies on measuring the relative change in resistance due to relative changes in developed strain caused by generated reaction forces upon engagement with tissue.

Rapid prototyping through 3D printing can significantly improve not only the product development life-cycle and design process by quickly prototyping design iterations but also address the long lead times required to develop the tools and fixtures necessary to fabricate components popularity due to its advantages such as ability to fabricate geometrically complex features, availability of variety of bio-materials, and ability to fabricate personalized devices that are safe and cost effective.

According to aspects of the present technology, the design, fabrication, and characterization of an encapsulated uniaxial micro-force sensor less than 3.5 mm in overall diameter are provided. This force sensor, in some instances, can be attached on the tip of a micro-robot capable of positioning and properly orienting the sensor in confined and hard to access areas of the body to evaluate tissue in vivo. The sensor can probe the tissue by controlled indentation and record the reaction and relaxation forces as function of time. The measured data can then be used to evaluate localized biomechanical properties of the tissue.

To address the challenges faced by conventional manufacturing processes and expedite the fabrication of design iterations for this technology, the components of the micro-force sensor were fabricated using 3D printing technology. One of the major challenges in fabricating the micro-force sensor using 3D printing was to meet geometric constraints while maintaining the desired performance specifications.

Described herein, the operational and design requirements or parameters for the sensor are provided. As will be appreciated, the force sensor described herein can also be referred to as a micro force sensor, miniature force sensor, sensor, and/or sensing element. Procedures followed for the design and fabrication of the sensing element will be described herein using traditional machining. The fabrication of various sensor components using 3D printing along with advantages and challenges faced using a Form3 printer will be further described. The procedures followed and tools developed to characterize the performance of the assembled sensor will be described. Subsequently, the development and use of a test bed to evaluate the in vivo operational performance of a sensor when interacting with a human forearm and the use of force relaxation data to evaluate biomechanical properties (viscoelastic constant) according to the Voigt model will be described herein.

Micro force sensor design. In vivo measurements for evaluating viscoelastic properties of organs such as the bladder or pelvic organ tissue are beneficial in that they provide for the quantitative assessment of the healthiness of the tissue. The design requirements of the micro-force sensor technology described herein must consider accessibility to the confined space site as it relates to its size, the range of the normal force to be applied at the tip of the sensor and operational environment conditions.

According to conducted studies, ideally an outside diameter of a flexible endoscope must be ≤7.4 Fr (2.4 mm) to avoid ureteric dilatation. In order to maintain a balance between the passage and durability of the device, the outer diameter of commonly used flexible endoscopes in adults falls within a range of 15Fr to 25 Fr (5.0 mm to 8.3 mm). The other force sensors designed for accessibility in confined space was reported to be 4 mm in diameter. Further other research has noted that a device with a limited contact duration (≤24 h), needs to be fully packaged and sterilizable to maintain biocompatibility.

The environmental constraints needed for the operation of a micro force sensor include accessibility to confined spaces in the human body, ability to operate in wet/moist conditions, and biocompatibility. The issues associated with biocompatibility and the moist operating environment of the bladder could be addressed by encapsulating the sensor with a biocompatible protective sheath or covering. One of the major constraints for the force sensor considering the intended application of bladder diagnosis was the sensor outside diameter which was preferred to be (≤5 mm).

Studies have shown that pelvic organs can undergo a reaction load of approximately 0.8-1.2 N with indentation depth ranging from 8-10 mm. Accordingly, a micro-force sensor as described herein must safely withstand a normal load of 1.2 N with a safety factor suitable for medical devices; according to the present technology a safety factor greater than 3 is used. The design requirements and aspects of the micro-force sensor are summarized in Table 1.

TABLE 1

| Characteristics | Specifications |
|---|---|
| Dimensions | Sensor housing diameter ≤3.5 mm |
| Force Range | Normal force ≤1.2N |
| Resolution | 20 mN |
| Safety Factor | ≥3 |
| Operating | Wet/Moist |
| environment | |
| [symbol] | Biocompatibility |

[symbol]

indicates data missing or illegible when filed

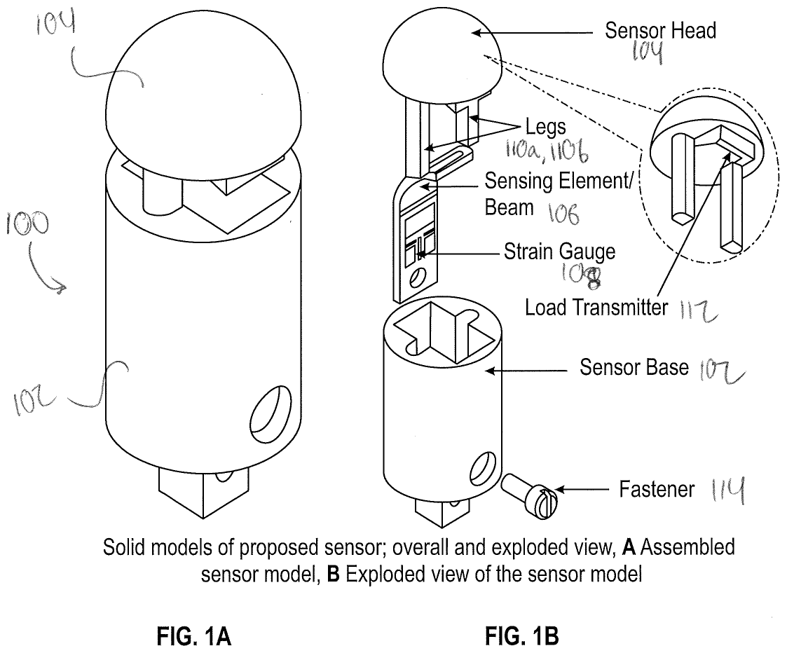

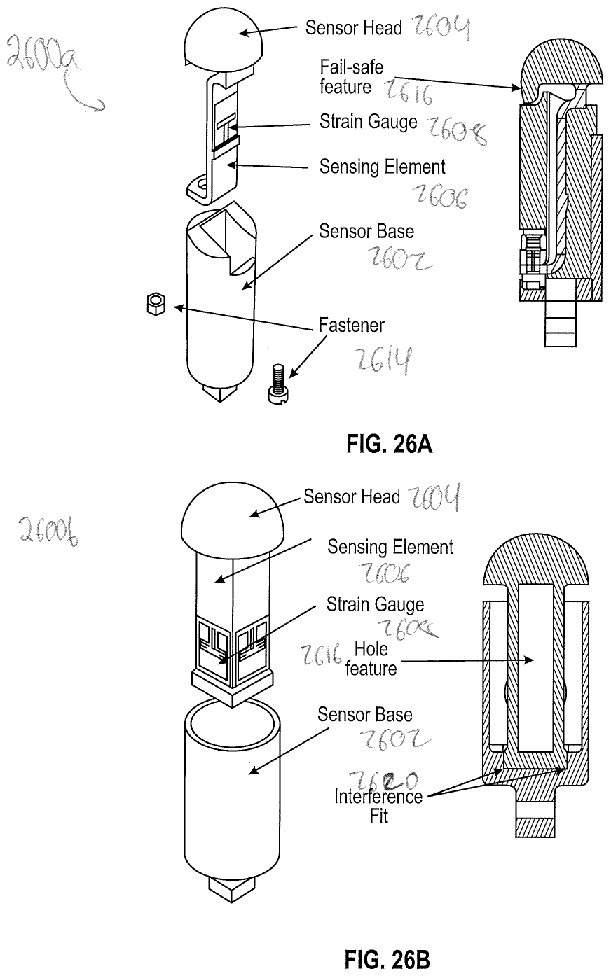

Sensor Concept. At a high level, various aspects of a micro-force sensor are described herein. Referring to the figures, a CAD model of an example sensor is illustrated in FIG. 1; accordingly, FIG. 1A is an assembled view of a micro-force sensor 100 and FIG. 1B is the exploded view of a micro-force sensor with an inset showing various other features of the sensor head. As will be appreciated, the biocompatible covering is not shown for visualization purposes in FIG. 1, but can be incorporated into any of the embodiments of a micro-force sensor. The nomenclature used for the sensor components generally follows the labels in FIG. 1B. Referring to FIG. 1A, a micro-force sensor 100 is illustrated, the micro-force sensor 100 comprising a sensor head 104 and a sensor body 102. Referring now to FIG. 1B, the sensor head 104 can comprise one or more legs 110a, 110b. As shown, the sensor head 104 can further comprise a load transmitter 112, the load transmitter 112 configured to transmit a force applied to the sensor head, which can be transferred to a sensing element/beam 106 and/or a strain gauge 108. As such, micro-force sensor 100 can further comprise a sensing element and/or beam 106 and one or more strain gauges 108. In some aspects, the strain gauge can be can be affixed to, in operable communication with, and/or integrated with sensing element 106. Turning briefly to FIGS. 26A and 26B, other embodiments of a micro-force sensor are depicted, for instance FIG. 26A illustrates another (monolithic) uniaxial micro-force sensor, and FIG. 26B illustrates a multi-axial micro-force sensor. As shown in FIG. 26A, micro-force sensor 2600a can comprise a sensor head 2604 and a sensor base 2602. The sensor head can comprise a sensing element 2606 which can be integrated with sensor head 2604. Sensing element 2606 can further comprise a strain gauge 2608. In some embodiments, sensor head 2604 can comprise a fail-safe feature which can be integrated with sensor head 2604. In some aspects, micro0froce sensor can comprise an attachment mechanism, such as a fastener, to attach the body or sensor base 2602 to sensor head 2604 via sensing element 2606. In some other embodiments, a strain gauge, e.g. strain gauge 2606, can have planar dimensions of about 1.19 mm×0.40 mm and a linear operating limit of about +600μϵ. Any strain gauge not inconsistent with the present invention may be implemented. Looking at FIG. 26B, a multiaxial micro0force sensor 2600b is illustrated. A micro-force sensor 2604 can comprise a sensor head 2604 and a sensor base 2602. Sensor head can further comprise a sensing element 2606, which can be affixed or integrated with sensor head 2604. Sensing element 2606 can further comprise one or more strain gauges 2608. Sensing element 2606 can further comprise a hole feature 2618 internally. Further, in some aspects a sensor head 2604 can be attached to a sensor base 2602 via an interference fit 2620.

The sensor can be positioned at the point of interest and oriented to be perpendicular to the tissue to be queried or otherwise evaluated. The hemispherical surface of the sensor head can engage with the tissue. When the sensor head comes in contact with and indents the tissue, a reaction force will be generated on the hemispherical surface of the sensor head and transferred to the sensing element/beam via the load transmitter on the sensor head. The legs of the sensor head can ensure a sliding motion relative to the sensor base. For an in vivo diagnostic operation, the sensor base could be attached on a micro-robot to gain access to the confined space. The deformation of the beam due to the applied load from the sensor head will generate strain in the beam (the other end of the beam is fastened to the sensor base). The fastener used to affix the beam to the sensor base may in some instances be a 0.5 mm Unified National Miniature (UNM) fillister head screw by Antrin Miniature Specialties Inc. (Fallbrook, CA). The sensing element design considered the allowable space in the sensor base cavity, while maximizing the strain sensed without undergoing plastic deformation.

The sensing element (e.g. micro force sensor) can further serve as a mounting structure for a metal foil strain gauge such as the N2K-06-S5024G-50C/DG/E3. by Micro-Measurements (Wendell, NC) with planar dimensions 1.9 mm×1.4 mm, which may be integrated or otherwise mounted and put in operable communication with the sensing element.

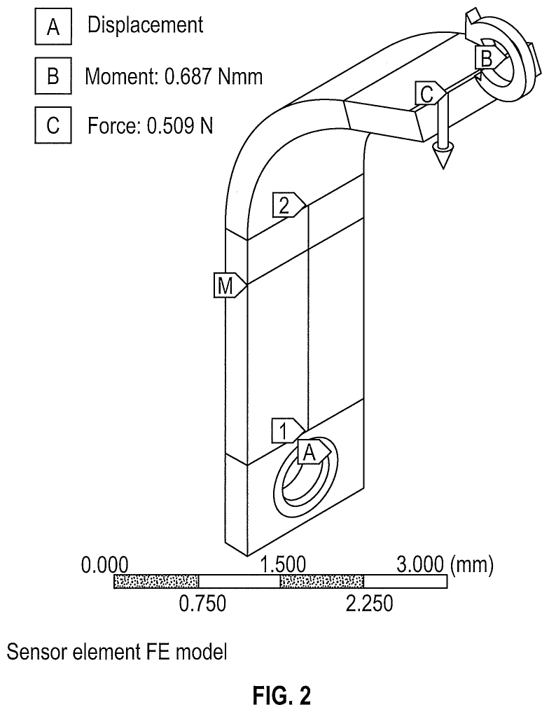

Finite element (FE) analysis was performed to identify the dimensions of the sensing element while maximizing the strain experienced due to applied load while remaining in the elastic region and meeting available space constraints. FIG. 2 shows the sensing element FE model with applied loads and boundary conditions. As shown in FIG. 1b, the load transmitter does not engage with the sensing element (beam) at the free end but rather on the curved part of the beam at a distance from the free end. This loading condition was modeled as an equivalent normal load and moment at the free end of the beam for the FE analysis. A zero displacement was defined at location A where the fastener will fix the beam element on the sensor base component. Location M defines the center of the active area of the strain gauge. The path from point 1 to point 2 (see FIG. 2) on the surface of the sensing element represents the path along which the strain will be evaluated since the strain gauge will be attached along this path. The Von-Mises stress, defined as the uniaxial tensile stress due to the distortion energy by actual combination of applied stresses was used as the failure criterion for designing components within the defined safety factor.

Sensor component fabrication. The size and complex features of the sensor head and sensor base components could not be easily fabricated using traditional machining processes. This created an impediment to prototyping several iterations of the sensor during design improvements and we investigated the use of 3D printing technology for fabrication.

3D printing served as the rapid prototyping platform for the sensor design due to the geometric features and size of its components without the need to fabricate custom fixtures and molds for traditional machining processes. 3D printing was also used to fabricate the fixtures needed for sensor characterization and experimentation. Fateri & Gebhardt discussed pros and cons of five 3D printing processes; Stereolithography (SLA), Selective Laser Sintering (SLS), Fused Deposition Modeling (FDM), Powder-Binding Bonding (3DP) and Layer Laminate Manufacturing (LLM).

As discussed by Ravi et. al., the mean dimensional error for complex geometric models of human organs fabricated using Form3B VP printer (Formlabs, Somerville, MA, USA) with a commercially available Grey material was 260 μm with good surface quality.

The fabrication specifications using a Form3 printer were well within the required feature size of the sensor housing components. Low Force Stereolithography (LFS), the fabrication process selected for prototyping the sensor components, is the 3D printing technology of the Form3 printer by Formlabs using Formlabs Grey material. The Grey material could be used to fabricate structures with a layer thickness measuring 25 μm as opposed to 50 μm and 100 μm with other Form3 compatible materials. The fabrication slicing paths were generated using the PreForm software by Formlabs.

### EXAMPLES

Sensor characterization. Having described various aspects of a micro force sensor and the fabrication thereof, the experimental setup and characterization of a sensor in accordance with the present technology is provided.

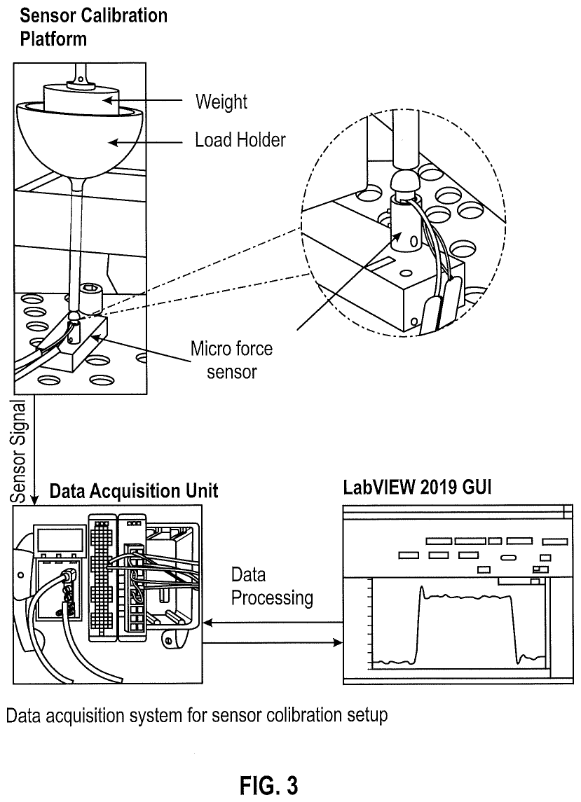

The characterization experiments were conducted using randomized experiments (to prevent biasing the results) with one factor (applied load) at five different levels. These experiments were designed to evaluate precision, sensitivity, resolution, and accuracy of the micro-force sensor as well as its calibration equation. The assembled sensor was set up on the calibration test platform, as shown in the upper left hand corner of FIG. 3.

The calibration test platform shows a load holder which applies the load in the normal direction on the sensor head. Sets of five dead weights were used for the calibration experiments of the micro-force sensor. The applied load would cause the beam and attached strain gauge to deform. The deformation in the strain gauge would generate a signal which was read by a data acquisition (DAQ) unit, a 24-bit NI-9219 (National Instruments Inc. Austin, TX) module, shown in the left hand bottom corner of FIG. 3. The NI-9174 chassis houses the NI-9219 module and was USB connected to a computer running LabVIEW by National Instruments. A Lab VIEW graphical user interface was developed to display and record the raw data acquired by the DAQ system. The resistance from the strain gauge was recorded with just the sensor head resting on top of the sensing element and found to be 5014.89Ω. This resistance will be referred to as the nominal or no-load resistance of the attached strain gauge. The application of a load resulted in a change in the resistance where a decrease in the resistance value indicated a compression load. The change in resistance, ΔRsg, nominal resistance, Rsg, and gauge factor, K, of the strain gauge were used to evaluate the equivalent strain due to an applied load according to Eq. 1:

$$ϵ = \frac{1}{K} \frac{Δ {R}_{s g}}{{R}_{s g}}$$

The calibration equation relating the measured strain, ϵ, (strain experienced by the strain gauge) to the applied load, F, and the calibration factor, Cf, is presented in Eq. 2:

$$ϵ = {C}_{f} \times F$$

The equivalent sensed load using strain measurements during the tissue properties experimentation was evaluated by re-arranging Eq. 2 to yield Eq. 3:

$$F = \frac{ϵ}{{C}_{f}}$$

The resolution of a sensor is defined as the smallest absolute change in resistance that could be detected by the measurement device. Sensitivity is the ability of the sensor to capture the smallest change in output variable (resistance) for a given input variable (applied load). Accuracy of the sensor is defined as the deviation of the measured quantity from the theoretically estimated value. The accuracy of the sensor is evaluated by comparing the strain evaluated from the measured resistance to the theoretical strain obtained from FE analysis. The error between the theoretical and measured strains is evaluated according to Eq. 4:

$$Error = \vert \frac{Theoretical strain - Experimental strain}{Theoretical strain} \vert \times 100 \%$$

Precision refers to how closely individual measurements are in agreement with each other for a particular loading condition. Precision is computed according to Eq 5., where Msd is the maximum deviation observed throughout the measurement and Avg(Msd) is the average measurement throughout the five sets of data for the particular loading condition. Eq. 5:

$$Precision = ( 1 - \vert \frac{{M}_{sd}}{Avg ( {M}_{sd} )} \vert ) \times 100 \%$$

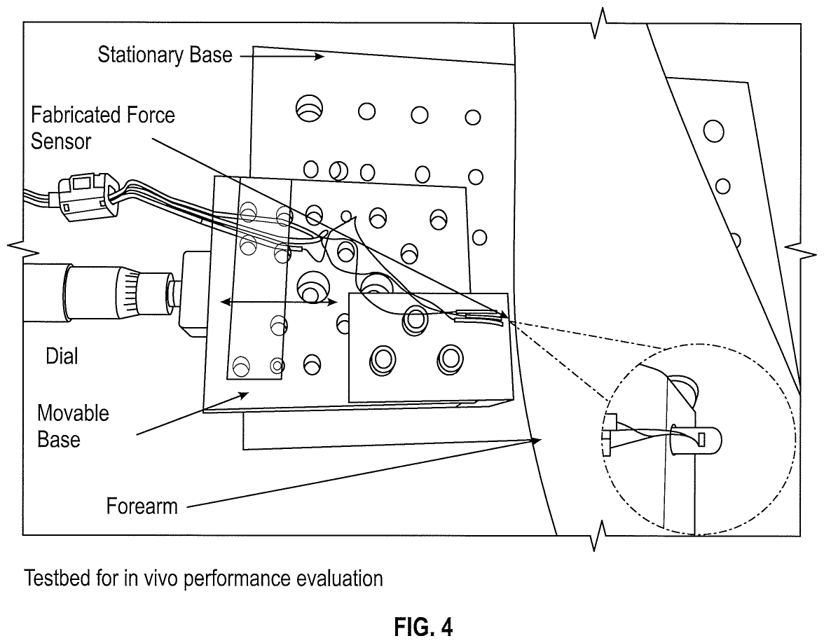

Sensor operational performance. The test bed shown in FIG. 4 was developed to obtain initial reaction strain data to validate sensor operational performance by measuring tissue relaxation, and then use the relaxation data to quantitatively characterize biomechanical properties of the interrogating tissue on a human forearm.

The test bed allowed for the sensor to be manually translated using a micrometer dial to a desired indentation distance. After indentation, the sensor was kept at this position for a predefined time while the tissue relaxed. The collected strain data as function of time were transformed into force using the developed characterization Eq. 3.

A number of different models have been proposed to evaluate biomechanical tissue properties such as Voigt model, Kelvin-Voigt model, Prony series, and Neo-Hookean. In this research, the transformed relaxation force data was used to identify viscoelastic properties of the tissue as function of the relaxation time according to the Voigt model. The Voigt model quantifies the ratio of the elastic constant to the damping coefficient as a function of time and quantification of this ratio helps to estimate viscoelastic property of the tissue. The solution to the Voigt model is given by Eq. 6 where f (t) is the measured reaction force response during tissue relaxation, fpeak is the peak reaction load sensed by the sensor, fresidual is the residual force, and τ is a coefficient representing the tissue recoil during the recovery phase. Eq. 6:

$$f ( t ) = ( {f}_{peak} - {f}_{residual} ) {e}^{- t / τ} + {f}_{residual}$$

Experimental results. The results of the analyses performed to design and fabricate the sensor are now presented. Also, various points of using the Form3 3D printer are highlighted. The fabricated and assembled sensor characterization performance matrix will be presented. Finally, the results of the in vivo forearm tissue characterization experiment will be presented.

The FE analysis results showed that the beam element experienced a Von-Mises stress of 35.9 MPa and 81.9 MPa near the mounting hole when loads equivalent to 510 mN and 1.2 N were applied respectively. A factor of safety of 3.4 was evaluated for the desired load capacity of 1.2 N using the Von-Mises stress criterion.

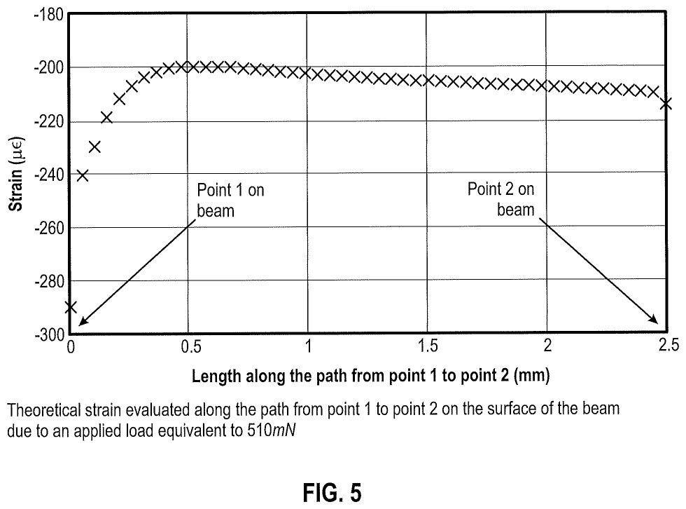

The strain experienced on the path between points 1 and 2 of the beam due to an applied load equivalent to 510 mN is shown in FIG. 5. The strain exhibited a linear behavior along a section of the path indicating that the active length of the strain gauge needed to be placed in this region. The 0 and 2.5 (units) on the horizontal axis represent points 1 and 2 on the sensing element respectively (see FIG. 2).

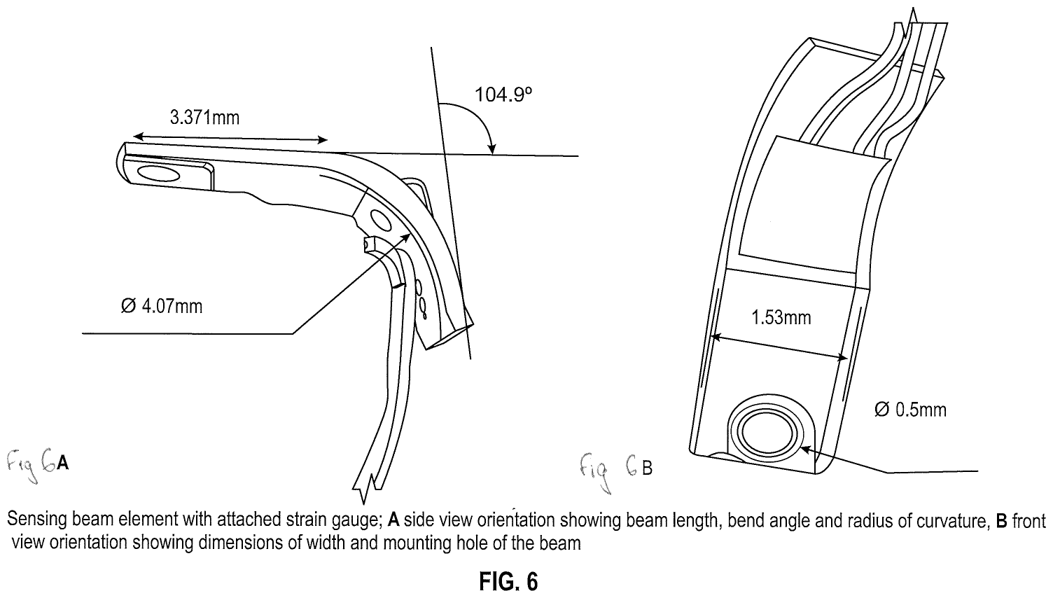

The sensing element was fabricated using a 0.3 mm thick aluminum foil cut using a shearing machine at the required dimensions established using FE analysis. A 0.5 mm mounting through hole was drilled at the designed location. The start of the bend radius was marked according to the design. A dowel pin with 4 mm diameter was used to achieve the designed bend radius for the aluminum strip at a desired bend angle of 100 degrees. The spring back effect of the material yielded a bend angle of 104.9 degrees. The fabricated beam element with an attached micro-strain gauge at the desired location is shown in FIG. 6. The assembled beam sensing element design parameters are measured using a Supereyes Microscope (Shenzhen, Guangdong, China) as shown in FIG. 6a and FIG. 6b.

The Form3 printer was used to prototype sensor components. The initial printing attempts were not successful due to the features and size of the components. The sensor head was printed in three different orientations and the sensor base in two different orientations in an attempt to fabricate defect free parts. The print time needed to fabricate ten parts (six sensor heads and four sensor bases) was approximately 210 minutes. The parts from the 3D printer were then cleaned using Form Wash cleaning station with fresh isopropyl alcohol (approximate time needed 60 minutes).

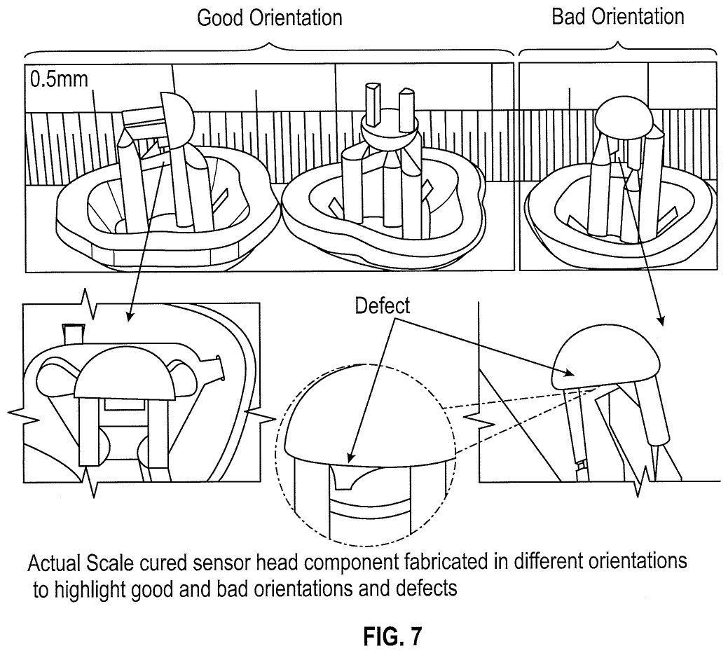

FIG. 7 shows cured sensor head components in three different orientations. The build orientation of the components was defined considering the alignment of the finished part with respect to the print direction. The 3D printed sensor head components were classified according to their print orientation as either being good or bad.

Sensor head components printed in a bad orientation exhibited defects at the load transmitter feature as shown in FIG. 7. The support structure was provided only at one end of the load transmitter feature, where the other end had no support which led to a failed part and thus a bad orientation. According to Formlabs fabrication specifications, the feature size (thickness) of an unsupported wall needed to be at least 0.2 mm. However, by printing the sensor head component with load transmitter feature thickness 0.2 mm in three different orientations exemplified a limitation of the fabrication specification for wall thickness. The incomplete fabrication of the load transmitter feature in the upright position was addressed by re-orienting the sensor head in the vertical upside down and/or side orientations and successfully fabricated the component as shown in FIG. 7.

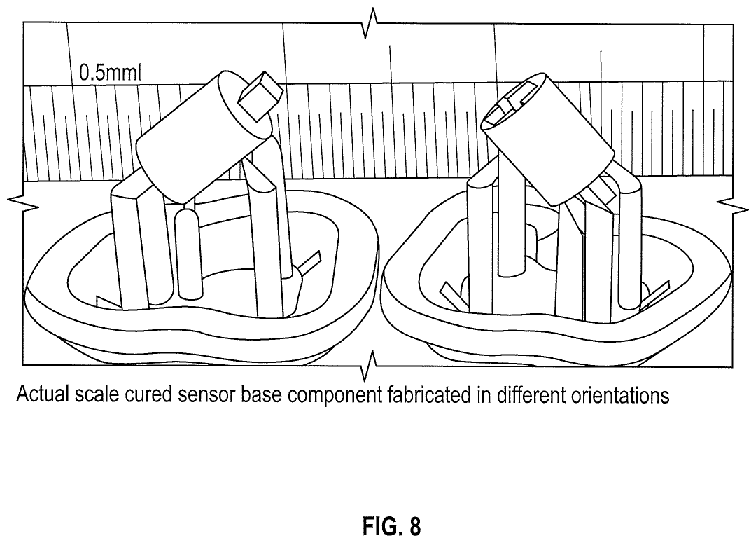

FIG. 8 shows cured sensor base components in two different orientations. The sensor base is a hollow cylinder with embedded features and identifying the print orientation was important to ensure that no polymer was clogged inside the hollow portion of the component during fabrication which might cause issues during cleaning and curing. Both build orientations of the sensor base component were acceptable since this component had a hole feature at the bottom face and a rectangular open face on the top and either of them could act as the drain to avoid clogging up material.

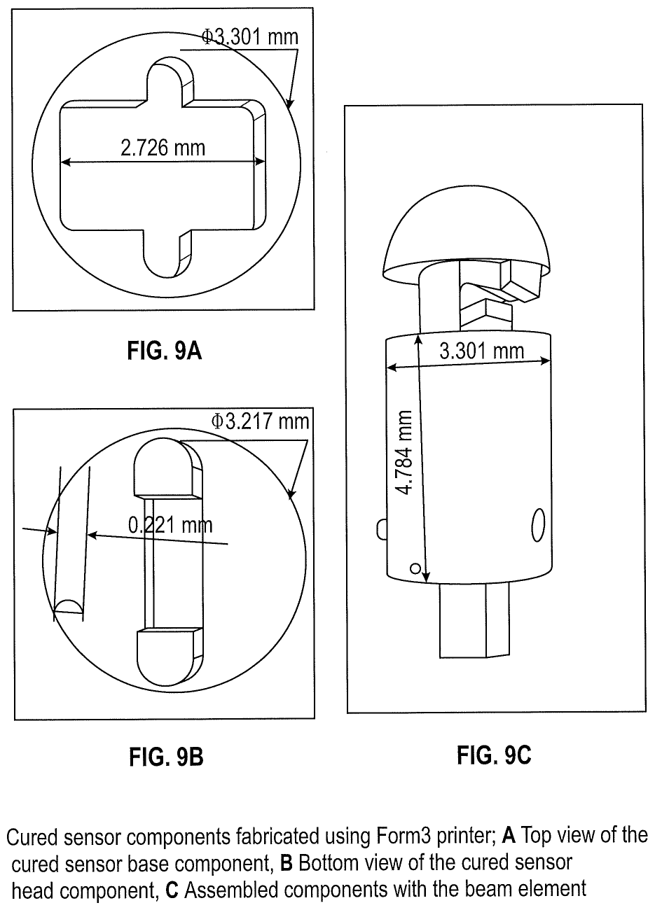

FIG. 9 shows the 3D printed and assembled sensor housing components with measurements using a Supereyes microscope. FIG. 9a is the top view of the sensor base component, less than the desired sensor housing diameter of 3.5 mm.

The rectangular slot in FIG. 9a was provided to accommodate the sensing element, where the semi-circular feature at either side of the rectangular slot are the guide ways for the legs of the sensor head component. FIG. 9b shows the bottom view of the sensor head component. FIG. 9c depicts the prototyped sensor housing assembly with the sensor head, sensor base and beam components.

The defect free components obtained from the good orientation of the print are shown in FIG. 7 (sensor head) and FIG. 8 (sensor base). The components printed in the good orientation were not only defect free but also had good dimensional accuracy. The comparison of dimensions of the as designed and as fabricated components is presented in Table 2.

TABLE 2

| | Designed | Fabricated* | Absolute |
|---|---|---|---|
| | dimensions | dimensions | Difference |
| Feature name | (mm) | (mm) | (μm) |
| Sensor base diameter | 3.400 | 3.301 | 99 |
| Sensor base height | 4.550 | 4.784 | 134 |
| Length of rectangular | 2.600 | 2.726 | 126 |
| slot on sensor base | | | |
| Sensor [symbol] diameter | 3.400 | 3.217 | 183 |
| Load [symbol] thickness | 0.200 | 0.221 | 21 |
| on sensor head | | | |

*measured using [symbol] system [symbol]
indicates data missing or illegible when filed

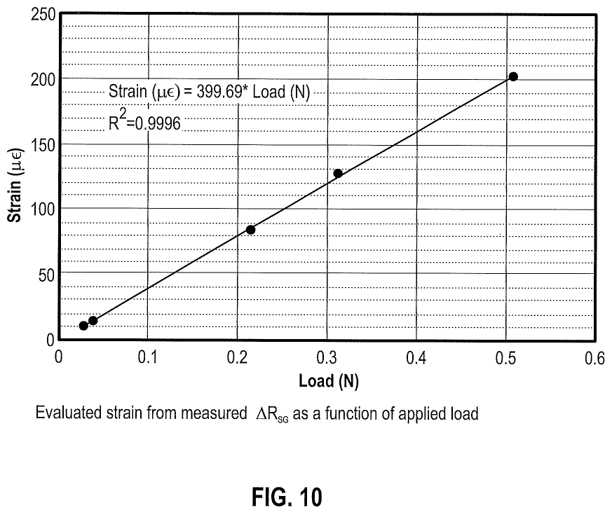

Sensor characterization. The sensor characterization experiments were performed using dead weights corresponding to applied loads of 28 mN, 37 mN, 214 mN, 311 mN, and 509 mN inclusive of the load holder weight of 18 mN. Each load test was performed in five randomized trials for a total of 25 experiments. The analysis of the sensor characterization provided insight on the behavior of the micro-force sensor system. FIG. 10 shows the calibration curve for the prototyped sensor in the force range of 0-509 mN.

Each data point in FIG. 10 represents the calculated mean strain from the measured sensor signal for a particular load after all the randomized experimental trials were completed. The line represents the curve fit for the calculated strain as function of the applied load. The characterization test indicates excellent linear behavior (R2=0.999) between the strain gauge sensor output and applied load. According to the curve-fit equation (see FIG. 10), the sensitivity of the sensor (strain/load) is evaluated to be 399.69μϵN.

In the load range of interest, the resolution of the data acquisition device was 3 mΩ, which corresponded to an equivalent load of 0.7 mN. The sensor response from the preliminary characterization revealed that the sensor was capable of sensing an applied change in load equivalent to 18 mN which was less than the 20 mN desired resolution. This indicated the hardware resolution capability as being 29 times better than the desired sensor resolution.

The experimental measurements and theoretical results were used to develop an accuracy matrix for the performance of the sensor for a subset of the runs which is presented in Table 3. The first column represents the experimental run order number out of 25 experiments. The second column is the load applied on the sensor head. The third column is the calculated change in resistance in the strain gauge used to estimate the experimental strain according to Eq. 1. The theoretical strain for each loading case was obtained from the FE model at location M (see FIG. 2).

According to Table 3, the largest error occurs at the smaller applied load of 28 mN. A minimum precision of 67% was evaluated at the lower applied loads reaching up to 92.6% for the higher loads, or up to 99% in some instances independently using Eq. 5.

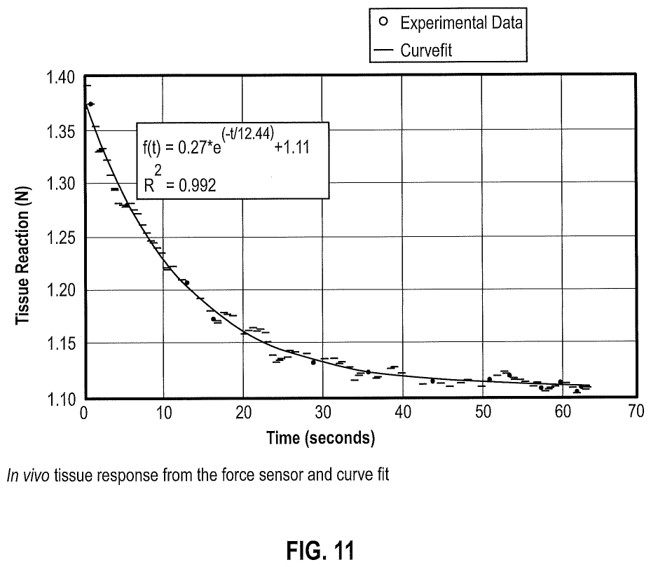

The in vivo operational performance of the sensor was evaluated using the developed testbed (see FIG. 4) on human forearm. After manually positioning the sensor at an indentation depth of 6.3 mm using the micrometer dial, the forearm was held fixed and the tissue allowed to relax. The strain measured by the sensor due to this indentation depth corresponded to an equivalent load of 1.37 N. The strain measurements from the sensor response as function of time were obtained using the same data acquisition setup used for the sensor characterization experiments. The strain measurements were post-processed according to Eq. 3 to obtain the relaxation force. The time data was also post-processed such that the relaxation state starts at time 0 sec. The post-processed time series relaxation force data used for evaluation of biomechanical tissue properties is shown in FIG. 11.

The viscoelastic time constant of the Voigt model was evaluated by curve fitting the relaxation data set according to Eq. 6. The analysis estimated a viscoelastic time constant of 12.44 sec with R2=0.992 with peak and residual forces of 1.37 N and 1.11 N respectively.

TABLE 3

| Run | Applied | ΔR_{SG} | Experimental | Theoretical | Error |
|---|---|---|---|---|---|
| Order | Load (mN) | (Ω) | Strain ( [symbol] ) | Strain ( [symbol] ) | (%) |
| 3 | 28 | −0.11 | 10.81 | 11.14 | 3.13 |
| 10 | 38 | −0.15 | 14.73 | 14.65 | 2.12 |
| 1 | 214 | −0.87 | 85.46 | 86.28 | 1.89 |
| 2 | 313 | −1.28 | 125.73 | 125.91 | 1.40 |
| 4 | 509 | −2.06 | 202.35 | 204.55 | 1.18 |

[symbol]

indicates data missing or illegible when filed

As is demonstrated herein, the fabricated micro-force sensor using 3D printing technology was able to provide high performance and that this sensor could further be used to characterize biomechanical tissue property. The fabrication using the Form3 printer required properly orienting the parts during preprocessing in the PreForm software. Properly orienting the parts was an important step, to assure the features of the components were fabricated according to the specifications provided by Formlabs. Additional experimentation was carried out by placing sensor components to be fabricated using the Form3 printer at different orientations to identify the preferred orientation to obtain quality parts without defects.

The dimensions of the features of the 3D printed components were in good agreement with the designed geometric specifications. The maximum dimensional error evaluated for the fabricated components was 183 μm for the sensor head diameter.

The performance of the sensor was evaluated using the assembled prototype. The characterization of the sensor performance using the developed testbed and dead weights demonstrated a linear behavior with a high regression coefficient. This indicated that the sensor design and identified dimensions of the sensing element allowed the sensing element to operate in the recoverable elastic region of the material without experiencing plastic deformation.

The comparison of the theoretical strain obtained from the FE analysis and the experimentally measured strain showed a maximum accuracy error level of ˜3% with the error reducing as the applied load increased. The error behavior, i.e. error reduction as load increases, could be attributed to easier overcoming frictional losses due to the sliding of the sensor head legs in the sensor base slot. Even though the maximum error was ˜3%, further investigation to identify the factors that contribute to this error at the small load values is warranted.

The in vivo experiment was performed on a human forearm. The data in the relaxation region was post-processed and used to evaluate the viscoelastic time constant for the forearm according to the Voigt model of 12.44 sec. The curve fit had a very high regression coefficient of 0.992. The sensor operation can further be verified by performing several experiments to evaluate the sensor performance matrix for different tissue types and different individuals.

Aspects of the present technology show the design, fabrication, assembly and characterization of a uniaxial miniature force sensor with design requirements of an overall diameter of less than 3.5 mm and a load bearing capacity of 1.2 N. The dimensions of the sensing element were identified using FE analysis considering performance requirements of resolution, maximum applied load, location of attached strain gauge for linear performance as well as size constraints.

3D printing provided several advantages compared to traditional machining processes such as rapid prototyping for miniaturized structures and features, fabrication of complex geometries and the ability to modify the design to meet the desired design specifications. Even though sensor components fabricated using the Form3 printer (inverted vat photopolymerization 3D printing platform) and Grey material exhibited good dimensional accuracy, load bearing capacity, function, and performance, identification of the correct fabrication orientation to obtain acceptable defect free components was determined.

The overall sensing performance of the sensor was experimentally assessed using dead weights for its sensitivity, resolution, accuracy and precision. The sensitivity of the sensor was 399.69μϵN. The sensor can sense reaction forces with a resolution of 18 mN. The sensor exhibited good accuracy by estimating reaction forces with a 3.13% error. The sensor was also able to maintain minimum precision of 67% at the lower applied loads reaching up to 92.6% for the higher loads independently.

The characterization of the sensor yielded a linear behavior with high regression coefficient. The characterized sensor was used for an in vivo experiment to evaluate operational performance using force relaxation data on a human forearm. The force relaxation data were used to find the viscoelastic time constant (τ=12.44 sec) according to the Voigt model. In addition, the curve fit of the Voigt model equation for the relaxation data yielded a very high regression coefficient (R²=0.992). Further, this type of micro-force sensor can be attached on delivery instruments to access confined spaces in the human body such as the bladder to interrogate tissue and use the measurements to evaluate quantifiable viscoelastic tissue properties over extended time periods for medical diagnosis and care.

According to some embodiments of the present technology, a sensor (or micro force sensor or miniature force sensor) for in vivo tissue evaluation and/or measurement is provided, and further embodiments of the technology is directed towards sensor(s) and sensing systems and methods of using the sensor(s) and systems for tissue evaluation, such as biological tissue. For example, sensor(s), systems, and methods can be implemented to evaluate viscoelastic properties of soft tissue, for instance as a part of a means to characterize a disease prognosis. As will be appreciated, identification of tissue viscoelastic properties can provide valuable information for assessing its healthiness or a disease state. Current technologies present challenges to access and perform localized tissue assessment in confined spaces in the human body through contact indentation/palpation. As such, there is a need for a diagnostic system capable of measuring tissue relaxation response at the local site by accessing the tissue through a natural orifice, or natural openings in the human body, while reducing patient trauma and improving comfort to palpate localized internal tissue surface anywhere, e.g. the bladder, and collect tissue relaxation responses. Of concern to medical practitioners is that current technology presents a gap to measure localized tissue relaxation forces in vivo.

According to some embodiments, a sensor (also referred to herein as a micro-force sensor), or further a sensing system, is provided that is a strain gauge based, uniaxial micro-force sensor. In some aspects a micro-force sensor is configured to measure tissue response data (e.g. biological tissue response) in confined human space environments. According to some aspects, a micro-force sensor can incorporate one or more components having specified component values. In one aspect, a micro-force sensor has an overall diameter of about 3.5 mm and can further have one or more specified characterization parameters. Such parameters can impart various performance characteristics to the micro-force sensor, such as load-bearing capacity, resolution, sensitivity, accuracy, precision, repeatability error, and hysteresis were evaluated to be 1.07 N, 0.13 mN, 859.7μϵ/N, ±28.6 mN, 87.2% (23 mN), ±3.13% (±25 mN), and 118 mN respectively.

According to some embodiments, micro-force sensors and methods of use or implementation can be provided for a confined human space application, for instance a human bladder which is a distensible hollow organ in the lower abdomen. As will be appreciated, other confined-space organs in the human body can be provided for application of a micro-force sensor. According to some embodiments of the technology, sensors and methods are provided that can obtain tissue relaxation responses of biological tissue through the controlled indentation in confined spaces, for instance in a human body. According to some further embodiments, micro-force sensors having the defined characteristics provided herein are configured to obtain force relaxation data from biological tissue to evaluate localized tissue viscoelasticity. Measuring such force relaxation response as a time history can provide useful information to evaluate changes in the tissue viscoelasticity as a function of elapsed time. Further, these changes could be an indicator of tissue disease or disease progression. Subsequently the results obtained through this can aid in the understanding of characterization of viscoelastic properties of soft tissue in vivo due to an indentation methodology.

According to some aspects, a micro-force sensor and/or sensing system are provided in accordance to one or more parameters or characterization factors described herein. In some embodiments, a micro-force sensor, e.g. a micro-force sensor, is configured to palpate tissue, for example the interior bladder wall tissue, for localized characterization of the viscoelastic properties of such tissue which can lead to improvement in medical diagnostic intervention.

According to some aspects, a methodology for the fabrication, assembly, characterization, and performance evaluation of a micro-force sensor is provided. According to some other aspects, a method for collecting force relaxation responses of soft tissue using a micro-force sensor is provided and characterizing the viscoelastic response of tissue using a linear solid model.

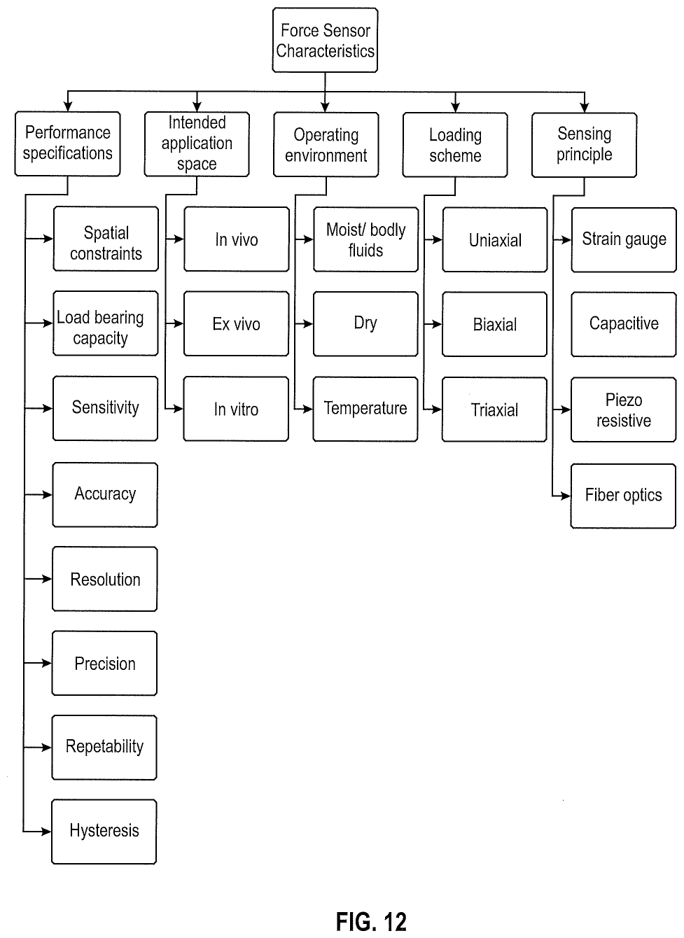

One application of a micro-force sensor provided herein is to palpate an internal organ tissue, and as such it is capable of measuring a reaction force of tissue. Mechanical inputs like weight, compression, pressure could be transformed as an output electrical signal, and the values of this electrical signal can be calibrated to obtain the equivalent value of the experienced force. The desired parameters of the sensing system can be identified by establishing the application and enumerating the required operational characteristics and are shown in FIG. 12.

These characteristics can be classified into five subcategories, performance specifications, intended application space, operating environment condition, expected loading scheme, and sensing principle. The conceptual design development of the micro-force sensor must emerge by considering at least one parameter from every category. The performance matrix of the sensor must be evaluated as it relates to the overall dimensions, load bearing capacity, sensitivity, accuracy, resolution, precision, repeatability, and hysteresis. Moreover, the desired performance specification matrix will provide guidelines for the development and identification of the data acquisition system. The application of the sensor is used to define information as it relates to the size of the sensor and the necessary precautions that must be taken to conduct the measurements safely. For instance, if the target tissue is inside the human body, it is generally necessary to consider the access point and the dimensions of the device to reach the desired organ, or tissue surface, within the human body.

Investigating the operating environment of the sensor can be used to define the encapsulation needed to protect the electro-mechanical subsystem in the sensor from its surrounding. For instance, if the sensor is envisioned to be operated inside of a human body, the sensor must be encapsulated with a bio-compatible protective sheath that will prevent the interaction of the bodily fluids with the force sensor components. As will be further appreciated, the structural design characteristics of a micro-force sensor according to the present technology considers various factors such as an expected loading scheme. For instance, an application of a micro-force sensor can be investigating tissue interaction forces while performing minimally invasive surgery, and as such, a force sensor that will be able to sense multiaxial loads can be beneficial.

According to some aspects, a micro-force sensor can be configured to interrogate the tissue to characterize its viscoelastic properties through normal-to-surface indentation or palpation; as such a uniaxial loading scheme will suffice this need. Selection of the sensing principle will assist in designing the deformable structure as well as defining operational and geometric constraints for the sensor development. The sensing principle will drive the selection and requirement for additional instrumentation needed to capture the data from the sensor. The micro-force sensor is configured to measure uniaxial loads using a metal foil strain gauge.

According to aspects of the technology, a micro-force sensor can be configured to meet certain specifications based on an application, for instance as used in a sensing system that can collect the internal organ in vivo tissue relaxation forces when palpated. In one aspect, the human bladder is the target organ for a micro-force sensor.

As such, the design specifications as they relate to the overall dimensions, expected force range, desired accuracy and resolution to effectively characterize the viscoelastic properties of the soft tissue are operational characteristics to be defined.

According to some aspects, one or more values for one or more critical parameters of a micro-force sensor can be determined. For example, a micro-force sensor must have a determined size as it relates to the ease of access for a designated confined space, the range of normal force applied to a micro-force sensor, a minimum required resolution, and one or more operational and/or environmental conditions.

With respect to dimensional constraints, in particular with respect to an application for locating a micro-force sensor in a human bladder, the dimensions of diagnostic devices need to be defined considering reported anatomical measurements. The average diameter with a maximum stretch of the external urethral meatus was reported to be within the range of 6.00 to 10.33 mm. As will be appreciated, previous findings strongly support the notion that using reduced-diameter transurethral instruments are directly correlated with minimizing and reducing patient trauma.

In one specific example application, e.g. applications in the pelvic region of a human, it is noted that organs proximal to the pelvic region undergo substantial deformations within the range of 5 mm to 8 mm due to small applied forces (0.5 N to 0.8 N). Accordingly, in some embodiments a micro-force sensor is configured to be able to sustain an equivalent load of 1 N with a safety factor acceptable in the medical device industry, and can comprise design specifications as shown in Table 4:

TABLE 4

| Characteristic | Specification |
|---|---|
| Diameter | ≤3.5 mm (sensor housing) |
| Force Range | ≤1.0N (normal force) |
| Resolution | 20 mN |
| Accuracy | ±30 mN |
| Safety Factor | ≥3.5 |
| Operating Environment | Wet/Moist |
| Other | Biocompatibility |

In some aspects, a micro-force sensor can be attached to a manipulator. As will be appreciated, manipulators composed of rigid links face constraints when it comes to effectively reaching portions of tissue to be measured, such as the entirety of the interior bladder wall for contact palpation. To address this limitation, a compliant manipulator with 6 degrees of freedom (DOF) distributed across 10 joints has been developed. This compliant design aims to extend the reach of the manipulator, with a desired position and orientation specifically targeting the ‘difficult-to-reach’ areas, for example within the bladder, including the trigone. According to some aspects of the present technology, a micro-force sensor can be attached to the tip of a compliant manipulator. The combined system, consisting of the manipulator and the attached sensor, has the potential to access the bladder and perform localized palpation and investigation of any area of the bladder interior wall. This can be accomplished by ensuring an appropriate load capacity and provide the ability to record the tissue response due to applied forces and use the information to evaluate tissue relaxation characteristics. In some aspects, a micro-force sensor can be employed for diagnostic applications, such as transurethral palpation or palpation during minimally invasive surgical interventions.

As will be further appreciated, a finer resolution will make it possible to capture tissue reaction response due to smaller applied forces. As will be appreciated, in some aspects, a micro-force sensor for in vivo tissue characterization can alternatively have a resolution of about 23 mN in order to capture tissue relaxation forces and subsequently characterize tissue viscoelastic properties.

In some embodiments, the uniaxial micro-force sensor consists of three major components; sensor head, sensor base and a sensing element. The sensor base component in some embodiments can be attached at the tip of a compliant manipulator. The sensor head can interact with the host tissue as the manipulator palpates and transfer the load to the sensing element/beam which will aid to sense/measuring the reaction forces. The sensing element can additionally function as a strain-measuring structure.

As previously noted, existing sensing technology has been analyzed and each of these technologies have drawbacks as it applies to interrogating the confined space environment in the human bladder. Investigating the benefits and drawbacks of fiber optics, piezo-resistive, and capacitive based strain measurement technologies it was identified that these sensing technologies could not be used because they do not meet at least one of the desirable specifications, relating to either size, accessibility to confined space environment and/or accessibility in the sensing structure itself. For example, dexterous manipulations are necessary to access and orient the manipulator in a confined space environment. Fiber optic-based sensing technology cannot operate reliably in confined spaces requiring a small bending radius with large bend angles which causes chirping losses and poor repeatability of measurements. Further it has been concluded that the sensitivity of a sensor and the bending of the optical fiber were directly correlated and attempting to measure reaction loads with a bend radius less than a few centimeters lead to erroneous measurements. In addition to performance limitations due to bend radius constraints, there is a further requirement of an amplification mechanism to improve the sensitivity, and challenges in implementing, routing, and moving the optical fiber through confined spaces in the human body. Piezo-resistive sensors on the other hand, have large overall sensor dimensions (4×4 mm2 and 9×9 mm2) and operational range limitations (1 mN and 30 mN). Strain gauge based sensing technology has been in use for many decades and they are reliable strain measuring devices. However, a miniature strain gauge must meet the desired spatial and operation constraints. According to some aspects of the present technology, a micro-force sensor in some embodiments considers positioning of the strain gauge in a location where the change in strain remains within its linear operating range.

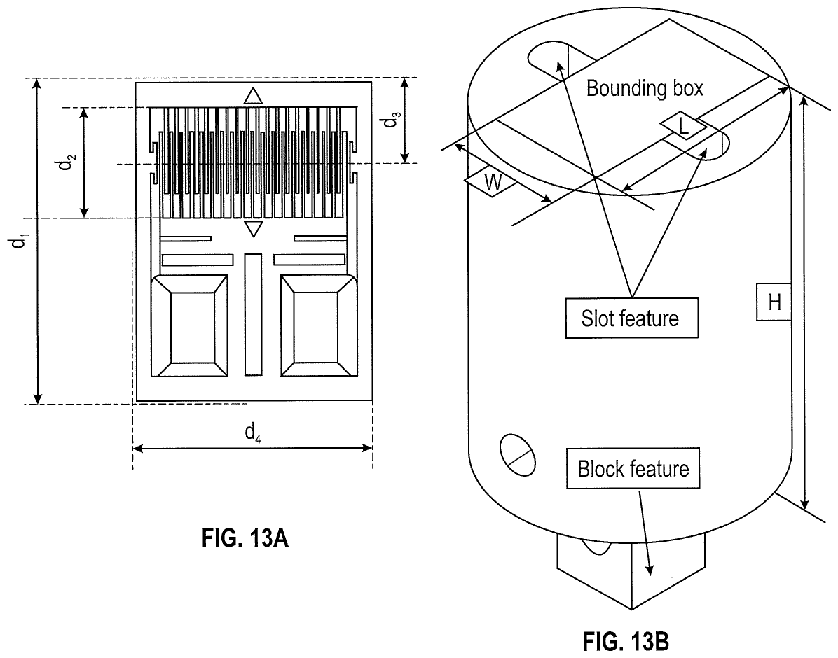

Since the overall diameter of the sensor is desired to be ≤3.5 mm, the size of the bounding box (See FIG. 13B) of the sensor base; a feature which will encompass the sensing element is defined to be 2.7 mm×1.55 mm×5 mm (length×width×height). Further increasing the length and/or width of the bounding box will require increasing the overall diameter of the micro-force sensor housing. As such the desired dimensions of the strain gauge must be less than the bounding box dimensions of 2.7 mm×1.55 mm×5 mm (length×width×height) of the sensor housing.

A miniature metal foil strain gauge with planar dimensions 1.9 mm×1.4 mm (N2K-06-S5024 G-50C/DG/E5, MicroMeasurements Inc., Wendell, North Carolina, US) can be utilized as meeting the determined dimensional constraints. The characteristic dimensions of the strain gauge can accommodate any desired application, and is shown in FIG. 13A, for instances, where d1=1.99 mm is the total length of the gauge packing, d2=0.48 mm is the gauge active length, d3=0.66 mm is the distance of the center of the active length of the gauge from the top, and d4=1.4 mm is the total width of the gauge packing. The linear operating range of the identified strain gauge is about +3000μϵ. One or more other strain gauges with similar characteristics not inconsistent with the technical objectives of the invention can also be implemented.

The sensing element serves as the mounting structure for the miniature metal foil strain gauge that is configured to measure the strain experienced by the sensing element during operation,

According to some embodiments a micro-force sensor is provided that can be implemented for tissue assessment in confined spaces.

According to some embodiments, a micro-force sensor is provided that enables better access to confined spaces of the human body. The novel design of the uniaxial micro-force sensor has an external diameter of ≤3.5 mm and 1 N load capacity for transurethral palpation of the bladder interior wall. The design of the micro-force sensor and a finite element (FE)-based discrete procedure to determine the values of the identified design parameters of bend radius, bend angle, and thickness while meeting defined operational and geometric constraints are presented. These values guided the prototyping of an aluminum sensing element with 2.18 mm bend radius, 104.9° bend angle, and 0.3 mm thickness. An experimental testbed was developed, calibrated and used for characterization experiments. The performance matrix of the prototyped micro-force sensor was experimentally evaluated. The sensitivity, resolution, accuracy, precision, and repeatability band of the sensor in some aspects are configured to be 859.73μϵ/N, 0.13 mN, 28.6 mN, 87.22%, and ±3.13%(˜±25 mN) respectively with a hysteresis of 118 mN.

According to aspects of the present invention, a micro-sensor described herein meets necessary design specifications towards reducing patient trauma and tissue damage and lesions while accessing organs through a natural orifice and performing measurements in vivo.

As such, devices and systems described herein are capable of accessing confined spaces in the human body to interrogate the tissue surface under consideration with an appropriate force range and resolution for meaningful measurements while substantially reducing patient trauma and discomfort during the procedure. Such a system must be capable to easily function in a clinical setting while the measurements could be easily performed and at regular intervals as prescribed by the physician. Rigid link manipulators have limited capability to interrogate the entire bladder through contact palpation, as such a 10-joint 6-DOF compliant manipulator was introduced to reach the ‘difficult-to-reach’ areas within the bladder including the trigone.

A micro-force sensor according to aspects of the present technology can in some instances attach to the tip of a compliant manipulator. The manipulator and attached sensor system can be used to access the bladder to palpate and interrogate any region of the bladder interior wall with appropriate load capacity while recording the tissue reaction and relaxation forces. The micro-force sensor can further be employed for diagnostic applications such as transurethral palpation or palpation during minimally invasive surgical interventions.

In some aspects, a uniaxial micro-force sensor is provided. In some example instances, the micro-force sensor is configured for transurethral palpation of the internal bladder wall tissue.

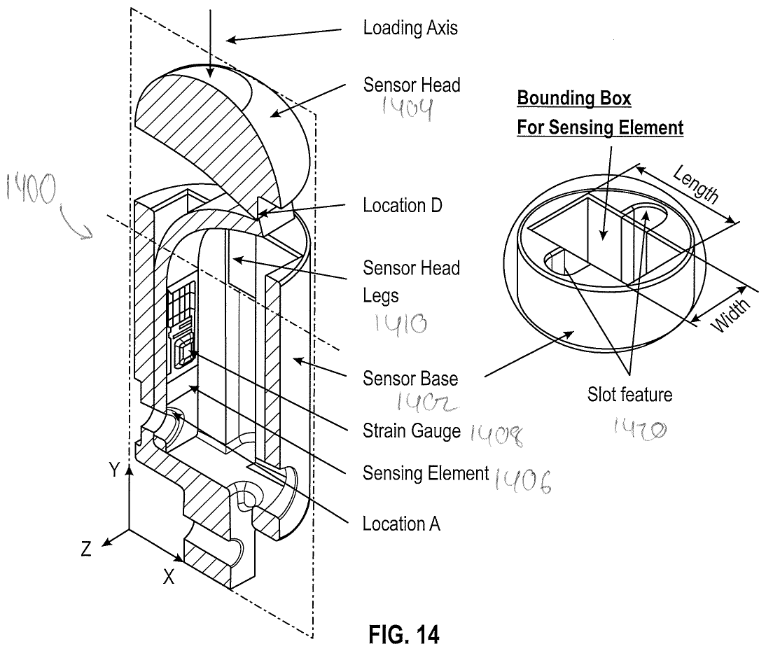

One example of a micro-force sensor in accordance with embodiments of the present technology is shown in FIG. 14. As shown, a micro-force sensor 1400 comprises a sensor head 1404, sensor base 1402, and sensing element 1406. In some aspects, sensor head 1404 comprises one or more sensor legs 1410. In some other aspects, micro-force sensor 1400 comprises a strain gauge 1408. In some instances, strain gauge 1408 can be affixed to, in operable communication with, and/or integrated with sensing element 1408. In some aspects, sensor base 1402 comprises a slot feature 1420, for example to receive sensing element 1406 and/or sensor head 1404, for instance by receiving one or more sensor head legs. The sensor base component can be in some aspects attached to a compliant manipulator. As the manipulator palpates the target tissue, the sensor head will translate along the loading axis (global Y-axis) and engage with the sensing element at location D as shown in FIG. 14. The load acting on the sensor head will be transferred to and generate a strain on the sensing element (since location A of the sensing element is fixed). The sensing element is configured to withstand the maximum applied load without undergoing plastic deformation while meeting space constraints. The measurements based on the sensing element behavior is also configured to meet a desired operational resolution and exhibit linear behavior in its range of operation while minimizing measurement error and hysteresis losses. Furthermore, micro-force sensors for confined space applications such as the human bladder must consider their size as it relates to ease of access through the urethra (reduce patient trauma and tissue lesions), the range of normal force to be applied at the tip of the sensor, and the operational environment conditions. In some embodiments, a micro-force sensor according to aspects of the present technology comprises one or more of the device specifications shown in Table 4.

The sensing element serves as the mounting structure for a miniature metal foil strain gauge to be used to measure the strain experienced by the sensing element. The strain gauge can be configured in some aspects to meet dimensional constraints and operational performance requirements. In one embodiments, the overall diameter of the sensor is provided to be about ≤3.5 mm, the size of an imaginary bounding box or space where the sensing element will reside (See FIG. 14) is defined to be 5 mm×1.55 mm×2.7 mm (height×width×length). Further increasing the length and/or width of the bounding box will require an increase in the overall diameter of the micro-force sensor.

Since the overall diameter of the sensor is desired to be ≤3.5 mm, the size of an imaginary bounding box or space where the sensing element will reside (See FIG. 14) is defined to be 5 mm×1.55 mm×2.7 mm (height× width×length). Further increasing the length and/or width of the bounding box will require an increase in the overall diameter of the micro-force sensor. The dimensions of the strain gauge impose additional constraints to the design parameters of the sensing element. The linear operating range of this strain gauge is ±3000μϵadds another requirement relating to its placement on a flat surface of the sensing element. The critical parameters affecting the strain experienced by the sensing element can also be implemented.

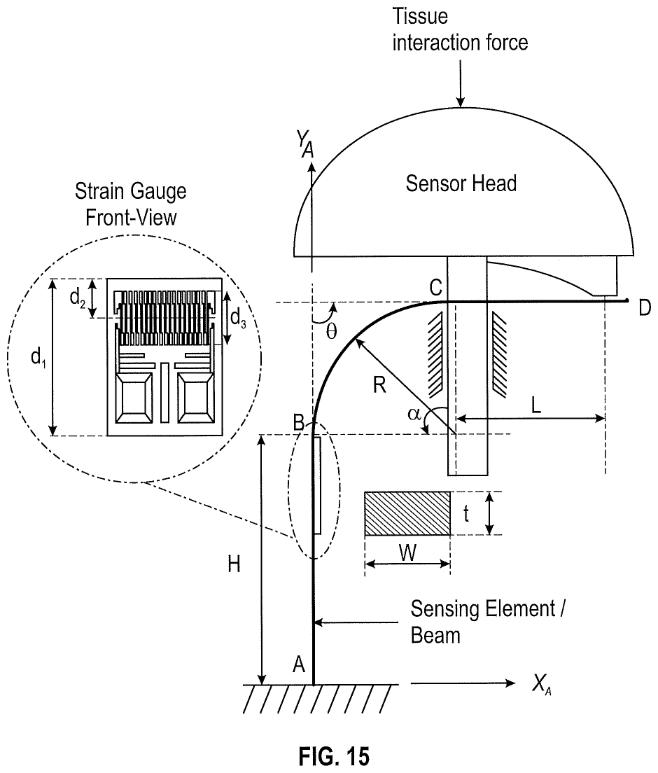

Micro-force sensor design. A schematic of an example micro-force sensor in accordance with embodiments of the present invention is shown in FIG. 15. The critical design parameters of the sensing element were defined and analyzed to maximize the absolute strain evaluated at the desired strain gauge attachment location in order to improve the sensitivity of the sensor. Since the strain gauge can be attached on a flat surface, the location will be along line AB, according to the example shown in FIG. 15. Considering the overall dimensions of the strain gauge, the height (length of segment AB in FIG. 2) of the sensing element must be at least 1.9 mm which is the total length of the strain gauge packing. In addition, for the fabrication of the active height of the sensing element, a provision for a mounting hole to attach the sensing element to the sensor base needs to be considered which requires the total fabricated height to be at least 3.9 mm.

The sensing element design parameters are the radius of curvature R, the bend angle relative to the vertical member of the beam θ, and the thickness of the beam t as shown in FIG. 15. Equation 7, shown below, demonstrates maximizing a strain of a micro-force sensor at a specified location on the sensing element while satisfying other defined constraints, where γ_{b}(R, θ, t) is the normal (Y-axis) strain evaluated at the desired location, γ_{sg} is the linear operating limit of the strain gauge, σₘₐₓ is the maximum von Mises stress experienced by the sensing element during loading, S_{y} is the yield strength of the sensing element material, N_{f}=σₘₐₓ/S_{y} is the factor of safety. Since this sensor is to eventually be part of a medical diagnostic device, the factor of safety is defined to be 3.50.

$$Max : ‖ {γ}_{θ} ( R , θ , t ) \vert μ ϵ ‖ \qquad ( Eq . 7 )$$

$$Such that : \{ \begin{matrix}{σ}_{max} \times {N}_{f} \leq {S}_{y} & [ MPa ] \\ R ( 1 + cos ( θ ) ) + L sin ( θ ) + \frac{1}{2} \leq 2.5 & [ mm ] \\ H + R sin ( θ ) - L cos ( θ ) \leq 6.5 & [ mm ] \\ {γ}_{sg} - \vert {γ}_{b} ( R , θ , t ) \vert \geq 0 & [ μ ϵ ]\end{matrix}$$

$$Limits : \{ \begin{matrix}0.2 \leq t \leq 0.35 & [ mm ] \\ 0.2 \leq R \leq 3.7 & [ mm ] \\ 90 \leq θ \leq 130 & [ deg ]\end{matrix}$$

One objective of the formulation of equation 7 is to maximize a strain at a specified location on the sensing element while satisfying other defined constraints of a micro-force sensor in accordance with embodiments described herein. As will be appreciated, the sensitivity of the sensor is directly related to the strain and as such maximizing the strain will improve the sensitivity of the sensor. However, the strain must remain in the linear operating range of the strain gauge of ±3000μϵ and the sensing material should remain in its elastic limit and not undergo plastic deformation.

The formulation for the sensing element must also consider where the central location of the active length of the strain gauge will be on the sensing element based on the characteristic dimensions of the identified miniature strain gauge. The characteristic dimensions are shown in FIG. 15; d1=1.99 mm is the total length of the gauge packing, d2=0.48 mm is the distance of the center of the active length of the gauge from the top, and d3=0.66 mm is the gauge active length. The dimensions of the strain gauge are used to define the candidate attachment location for its central axis at 0.48 mm from B towards A (See FIG. 15).

The maximum strain formulation includes inequality constraints for the allowable stress while geometric constraints are imposed on the overall dimensions of the sensing element. An inequality constraint is added to ensure that the estimated strain on the sensing element does not exceed the operating range of the strain gauge. The upper and lower bounds of the design parameters are defined using different criteria including geometric limitations, machinability, manufacturability, ease of assembly, improved sensitivity, and component availability.

The location of the contact point D along the bending element arm, CD, changes dynamically depending on the magnitude of the applied force and is function of (R, θ, L, t). This location causes the behavior of the bending element arm (part of the sensing element) to be a nonlinear function of the sensor head displacement.

A design point is defined as a unique combination of discrete design parameters of the sensing element; bend radius R∈[0.2, 3.7]mm in increments of 0.5 mm for 8 values, bend angle θ∈[90°, 130° ] in increments of 10° for 5 values, and thickness t∈[0.2, 0.35]mm in increments of 0.05 mm for 4 values. This discretization yields a total of 160 unique design points.

Out of these 160 design points, 31 were discarded since they did not meet the desired bounding box constraint (See FIG. 14 and Eq. 7). The remaining 129 design points, called feasible design points, were analyzed using parametric finite element analysis.

In some aspects, a micro-force sensor unit can be covered with a biocompatible sheath which addresses the environment in which the sensor will operate and allows for the use of non-biocompatible materials for the sensing element. In some embodiments a sensing element of a micro-force sensor comprises aluminum.

Finite Element Analysis. The part of the sensor head interacting with the sensing element was modeled to automatically align at the desired interaction location (location D in FIG. 15) on the sensing element through the geometric constraints presented in equation 7. The design parameters of the feasible design points were parameterized in SolidWorks™ (Dassault Systemes, Waltham, Massachusetts, US) CAD modeling software. The solid model was imported into the finite element program ANSYS™ (Canonsburg, Pennsylvania, US) Workbench R21.1. The imported CAD model was further processed to automate the search discrete characterization procedure by defining the design parameters in Ansys™ DesignModeler R21.1.

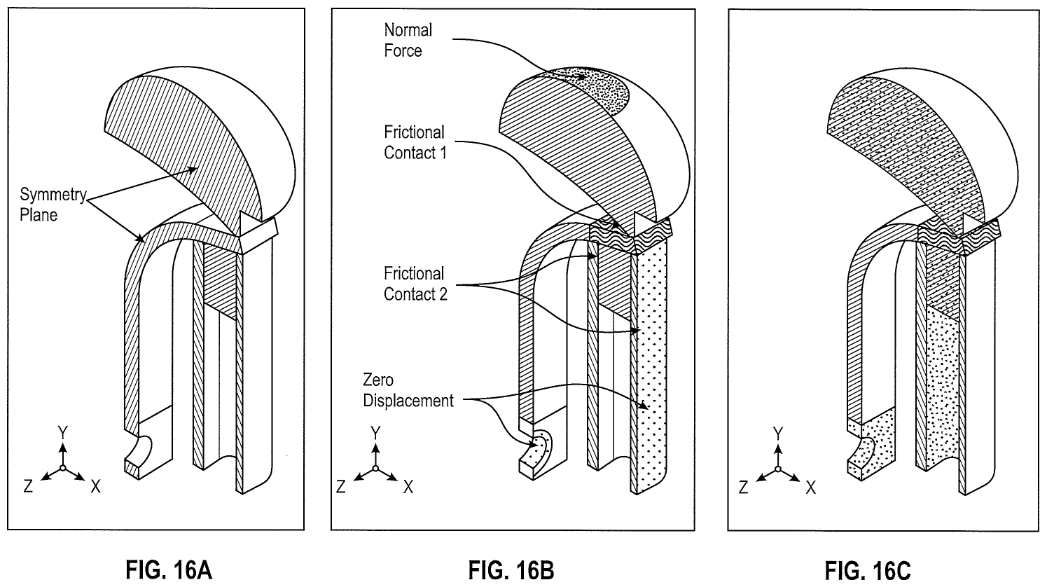

A planar symmetry boundary condition was advantageously employed (See FIG. 16A) to reduce computation time. The finite element model of the sensing element was pre-processed to obtain targeted and localized solutions and improve computational time. The interaction between the slots of the sensor base and the legs of the sensor head was also modeled by defining a frictional contact between them. The complex features of the sensor head and sensor base component were fabricated using additive manufacturing process. As will be appreciated, any fabrication process not inconsistent with the technical objectives of the present technology may be employed, for instance a fabrication process can comprise an injection molding process, an extrusion process, and/or a thermoforming process, for example.

The material properties of Formlabs Inc. (Somerville, MA, US) grey resin were used for structural analysis of the sensor head and the sensor base. The material properties of aluminum were assigned to the sensing element. The analysis for a single design point was performed by setting the boundary conditions for the developed model as presented in FIG. 16B. As will be appreciated, any other material or combination of materials not inconsistent with the objectives of the present invention may be used for the sensing element.

The FE model mesh was generated using 10-node tetrahedral and 20-node hexahedral elements, as presented in FIG. 16C. These elements improve the convergence rate since they offer robust adaptivity during mesh refinement, and to obtain higher solution accuracy due to their ability to handle complex geometric features for a computationally efficient solution. The adaptive mesh control process automatically iterates and refines the mesh density until either the convergence criterion (1%) or the maximum number of refinement loops are met. A representative mesh is shown in FIG. 16C.

The sensor is expected to sense normal palpation reaction loads up to 1 N. This load is used for design purposes to estimate the maximum stress and the safety factor of the sensing element. The load applied on the top surface of the sensor head during FE analysis is 0.5 N due to model symmetry as shown in FIG. 16B.

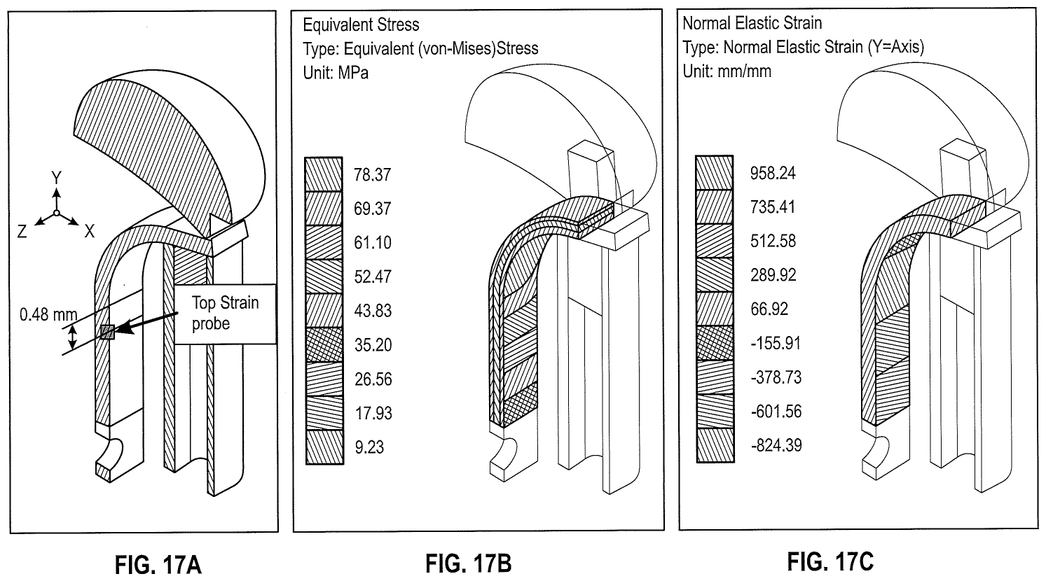

The normal strain along the Y-axis was evaluated at the desired location of 0.48 mm below the starting point of the bend which is indicated as ‘Top strain probe’ in FIG. 17. This location was selected considering the central axis of the active length of the identified strain gauge with respect to the strain gauge backing.

The von Mises stress distribution was evaluated for the targeted region of the sensing element. FIG. 17B shows the results of the von Mises stress distribution due to the maximum loading condition for a single design point by setting the design parameters bend radius, bend angle, and thickness to 2.2 mm, 110°, and 0.3 mm respectively. FIG. 4b shows that the maximum stress is located at the bend radius of the sensing element. Similar stress contours were observed for all design points. The maximum von Mises stress calculated for each design point is used to evaluate the safety factor for the respective design point.

For example, a safety factor Nᵣ=3.6 is achieved for the results presented in FIG. 17B, where the maximum von Mises stress was evaluated as σₘₐₓ=78.37 MPa and the yield strength of the material (Al-6061-T6) is S_{y}=280 MPa. FIG. 4c presents the strain distribution for the same design point (R=2.2 mm, θ=110°, t=0.3 mm) due to the maximum applied loading condition. The strain distribution contour aids in identifying the location to align the central axis of the active length of the strain gauge. Furthermore, it can be deduced from FIG. 17C that the maximum compressive strain was found near the top strain probe, indicating that the active surface of the strain gauge must be positioned near the top strain probe to obtain improved sensitivity. The strain evaluated at the top strain probe and the von Mises stress due to maximum loading conditions are set as output parameters. The results from the parametric analysis are post-processed to identify the optimal design parameters of the sensing element.

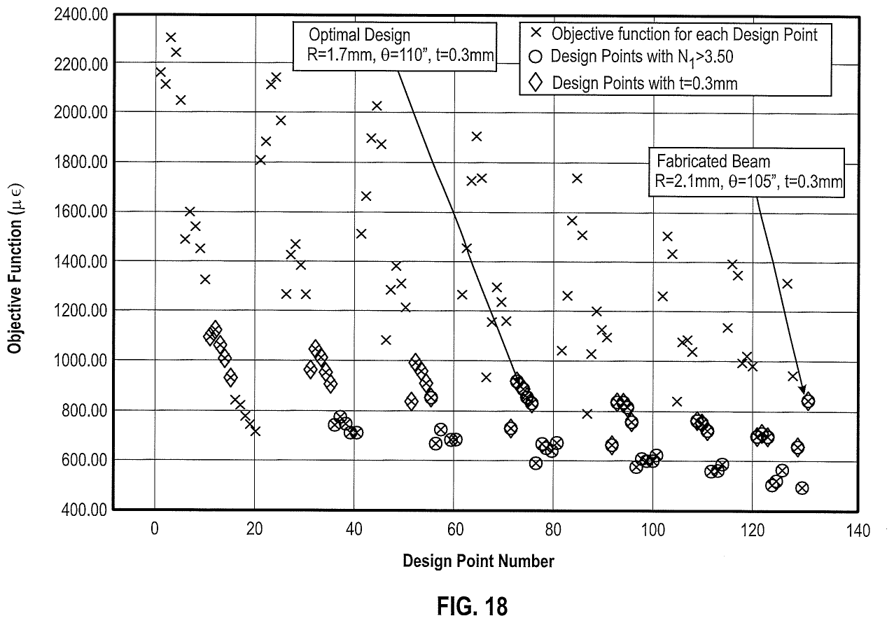

One goal in this example is to identify the geometric parameters which maximize the cost of the objective function (absolute value strain at the top strain probe). The cost of the objective function evaluated for the 129 feasible discrete design points is plotted in FIG. 18. The cross, (x), represents the cost of the objective function for all the feasible discrete design points. The circle, (○), represents those points exhibiting a factor of safety greater than 3.50. The diamond, (⋄), represents the design points with a beam thickness of 0.3 mm. One exemplary design point (point 73) has a bend radius of 1.7 mm, a bend angle of 110° and a thickness of 0.3 mm yielding an objective function cost and safety factor of 894.98μϵ and 3.54 respectively.

All designs with beam thickness ≤0.25 mm exhibited a factor of safety less than 3.5 and these design points were not considered for further analysis. Design point 130 in FIG. 18 presents the cost of the objective function evaluated using the measured dimensions of the fabricated sensing element. The cost of the objective function for the manufactured sensing element was evaluated to be 840.55μϵ, while the factor of safety was determined to be 3.74.

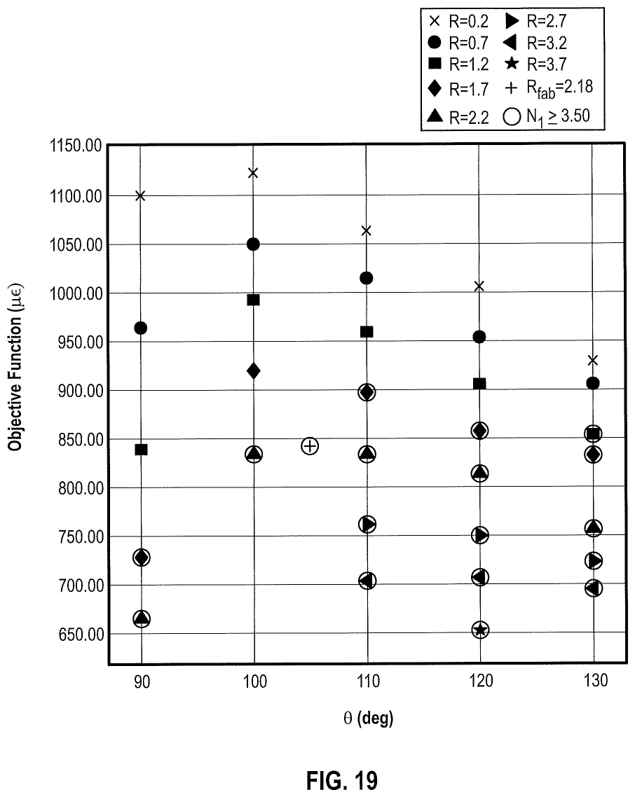

FIG. 19 shows the effect of bend radius and bend angle on the objective function when the beam thickness is set to 0.3 mm for the 32 feasible design points. As the bend angle increases from 90° to 100° the cost of the objective function increases for all bend radii however, further increasing the bend angle reduces the cost of the objective function. One exemplary design point in FIG. 19 is shown along the 110° bend angle with a bend radius of 1.7 mm and cost function of 894.98μϵ. As will be appreciated, if the bend radius increases to 2.2 mm or higher, then all feasible design points yield safe designs. This observation could prove useful during fabrication of the sensing element considering manufacturing constraints.

Fabrication of Micro-force sensor. A 0.3 mm thick aluminum sheet was used to fabricate the sensing element. A $4.00 mm dowel pin will generate a bend radius of 2.15 mm at the neutral axis for a 0.3 mm thick aluminum sheet. According to FE-based analysis, a bend radius of 2.15 mm yields a safe design with an objective function of 833.40μϵ when the bend angle is 100°. A 3-point bend press with an off-the-shelf 90° die was used to bend the aluminum sheet which will experience spring back after bending. These specifications and conditions even though not the optimal ones, were selected for the fabrication of the sensing element due to the availability of fabrication resources. The spring back affects the final bend radius Rf (mm) which can be calculated according to equation 8 below as a function of the initial bend radius Ri(mm), the material yield strength S_{y}(MPa) and modulus of elasticity E(MPa), and the thickness of the sheet metal t (mm).

$${R}_{f} = {[ \vert 4 {( \frac{{R}_{i} {S}_{y}}{Et} )}^{3} - 3 ( \frac{{R}_{i} {S}_{y}}{Et} ) + 1 ]}^{- 1} \cdot {R}_{i} \qquad ( Eq . 8 )$$

The length of the neutral axis of a curved member, L_{b}, also called bend allowance is a function of the inner bend angle α(rad), bend radius R(mm), thickness of the sheet metal t, and spring back factor k (k=0.5 for R>2t) shown in equation 9. The length of the neutral axis remains the same before and after elastic bending, which is used to solve for the final inner bend angle α_{f} (rad) as function of the initial inner bend angle αᵢ(rad) as shown in equation 10.

$${L}_{θ} = ( {R}_{i} + kt ) \cdot {α}_{t} = ( {R}_{f} + kt ) \cdot {α}_{f} \qquad ( Eq . 9 )$$

$${α}_{f} = \frac{{R}_{i} + kt}{{R}_{f} + kt} \cdot {α}_{i} \qquad ( Eq . 10 )$$

An expression for the final bend angle θ_{f} is obtained by combining equations 9 and 10 to yield equation 11.

$${θ}_{f} = π - {α}_{f} = π - ( \frac{{R}_{i} + kt}{\frac{{R}_{1}}{4 {( \text{?} )}^{3} - 3 ( \text{?} ) + 1} + kt} ) \cdot {α}_{i} \qquad ( Eq . 11 )$$

$$\text{?} \text{indicates text missing or illegible when filed}$$

Using equation 11, the final bend angle of the sensing element due to spring back is calculated to be θ_{f}=97° when the initial bend angle θi=90° and the initial bend radius Ri=2.15 mm for aluminum with yield strength Sy=280 MPa and modulus of elasticity E=68.9 GPa.

A sensing element, or fabricated sensing element design parameters were found to be an inner radius Rf-fab=2.03 mm with a neutral axis bend radius Rneutral-fab=2.18 mm, bend angle θf-fab=104.9°, and thickness tfab=0.3 mm. These parameters were used to perform a FE analysis with a load of 0.5 N (using the symmetric model) yielding an objective function cost of γb(Rf-fab, θf-fab, tfab)=840.55μϵ and factor of safety Nf=3.74(>3.5). These results are shown as the plus symbol (+) in FIG. 19. The sensor housing subsystem was built using additive manufacturing technology (low force inverted vat photopolymerization). The external diameter of the prototype sensor housing used in the present example is 3.3 mm.

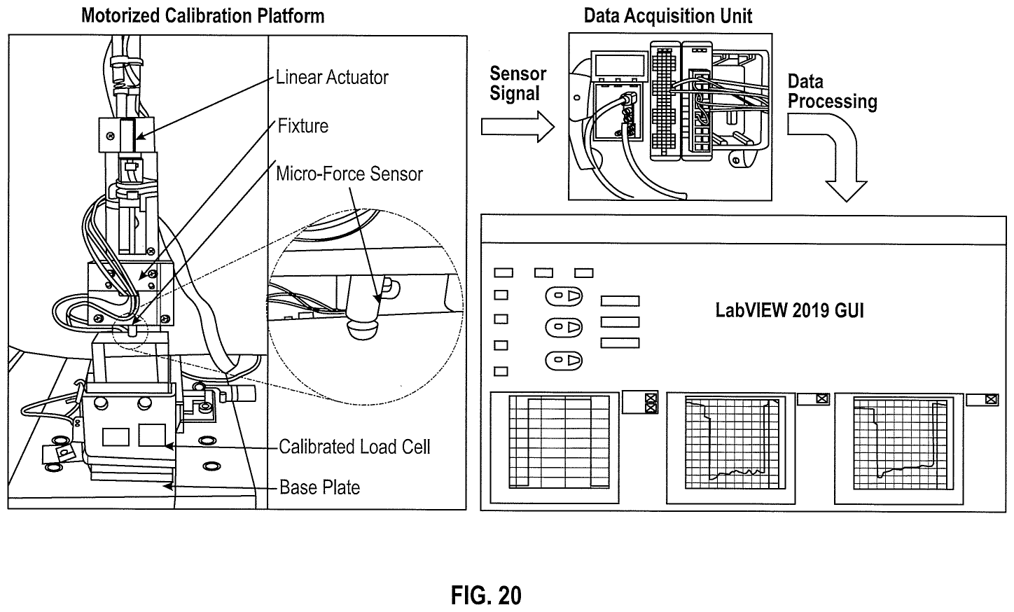

Micro-Force Sensor Characterization. An assembled micro-force sensor using components described herein was calibrated and its performance matrix developed. The performance matrix is based on post-processing the measurements from the sensor characterization experiments. Results from characterization experiments were compared with those obtained from the FE analysis. The experimental testbed is shown in FIG. 20.

The sensor-attached fixture mounted on the linear actuator was initially adjusted to a relative no-load state. A precision miniature translational stage (MM-4M-EX, National Aperture Inc., Salem, NH, US) with an accuracy of +2 μm was used for the controllable displacement of the micro-force sensor. A custom control and data acquisition program was created in LabVIEW™ (National Instruments Inc., Austin, TX, US) to control the DC servo motor using an NI myRIO microcontroller. A SMT series (4501017/B, MTSR, Eden Prairie, MN, US) load cell was calibrated using dead weights and then used to measure the reaction load from the sensor during controlled displacement loading.

The signals generated by the sensor and the load cell were acquired by a 24-bit National Instruments, NI-9219 data acquisition module (DAQ), mounted on a NI-9174 chassis connected to a computer running National Instruments Lab VIEW™ software via USB connection. Lab VIEW was programmed to acquire the load experienced by the calibrated load cell in newtons (N). A four-wire resistance measurement approach was implemented to measure the resistance of the strain gauge. The nominal resistance of the strain gauge attached to the beam was recorded as 4955.04Ω. A 24-bit DAQ device discretizes a 10KΩ (automatically selected range based on the nominal strain gauge resistance according to DAQ manual) into 1.19 mΩ discrete steps (10000/2²³). An equivalent strain γ_{sg} can be calculated as a function of the change in resistance ΔR_{sg}, the gauge nominal resistance R_{sg}, and the gauge factor K_{sg}, according to equation 12. The selected miniature strain gauge (N2K -06-S5024G-50C/DG/E5, MicroMeasurements Inc., Wendell, NC, US) has a gauge factor of K_{sg}=2.03.

$${γ}_{sg} = \frac{1}{{K}_{sg}} \frac{Δ {R}_{sg}}{{R}_{sg}} \qquad ( Eq . 12 )$$

Based on the resolution of the data acquisition device, the smallest measurable resistance change of 1.19 mΩ corresponds to a calculated strain change of 0.118μϵ. According to the FE analysis, a 10 mN(˜1.01 gram) load generates a strain of 13.13μϵ which, according to equation 12, corresponds to a resistance change of ΔR_{sg}=132 mΩ at the desired location. This resistance change is ˜111 times larger than the resolution of the measurement device, thus confirming the data acquisition device is suitable for sensing a minimum load of 10 mN.

The sensor characterization setup presented in FIG. 20 was used to perform the calibration.

The sensor characterization procedure involved randomized ordered loading and unloading experiments in triplicates with a single factor (applied displacement) at 15 distinct levels ranging from 21 μm to 315 μm at an interval of 21 μm. The experimental results were processed to evaluate the sensitivity, resolution, accuracy, precision, repeatability, and hysteresis of the sensor, i.e. the micro-force sensor.

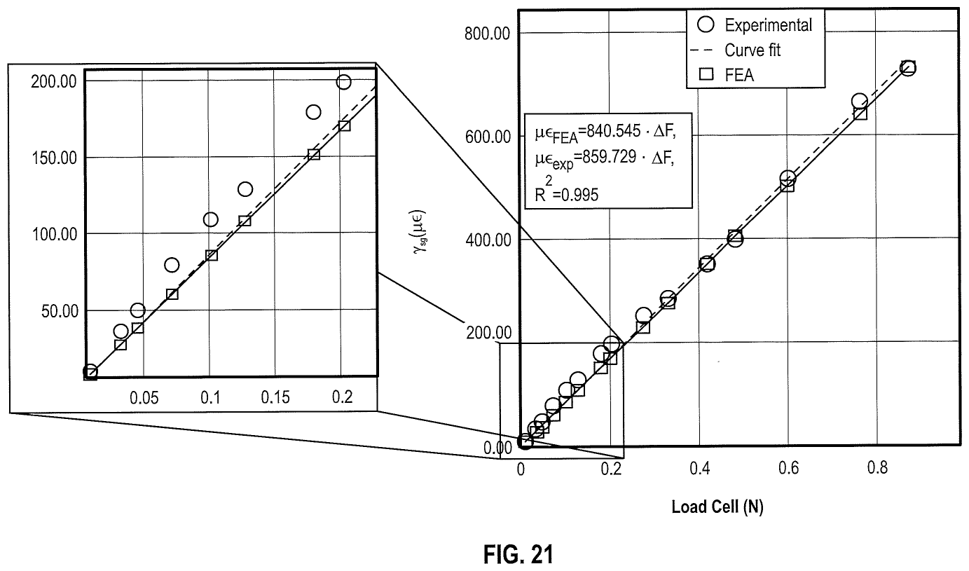

Sensitivity. Sensitivity is the ability of the sensor to capture the change in output variable (resistance) for a given change in input variable (applied displacement). FIG. 21 shows the sensitivity graphs for post-processed experimental strains evaluated from measured sensor signal using equation 12 and FE-evaluated strains. During the characterization experiments, the change in load from the calibrated load cell and the change in resistance from the strain gauge attached micro-force sensor were recorded. The calibration equation of the micro-force sensor is shown in equation 13, where ΔRsg is the measured change in resistance from the micro-force sensor, Co is the experimentally evaluated calibration factor, Ksg=2.03 is the strain gauge factor provided by the manufacturer, Rsg=4995.04Ω is the measured no load nominal resistance of the sensor strain gauge after attached on the sensing element, and ΔF is the force experienced by the micro-force sensor which is measured by the load cell.

$${γ}_{sg} = \frac{1}{{K}_{sg}} \frac{Δ {R}_{sg}}{{R}_{sg}} = {C}_{0} \cdot Δ F \qquad ( Eq . 13 )$$

The experimental and FE sensor calibration factor was found to be C₀₋ₑₓₚ=859.7μϵ/N and C_{0-FE}=840.55μϵ/N respectively with the sensitivity plots presented in FIG. 21. The calibration analysis shows an excellent linear behavior with a coefficient of determination R²=0.99 between the strain gauge sensor and the load experienced by the load cell. Thus further confirming that the force experienced on the sensor head can be accurately monitored and measured.

Resolution. The resolution of the micro-force sensor system is its ability to detect the smallest measurable change in the strain gauge resistance assuming the sensing element material is in its elastic limit and the attached strain gauge in its operating range. As established herein, the resolution of the data acquisition system allows for the measurement of a change in the resistance of the strain gauge of 1.19 mΩ which corresponds to an equivalent load change of 0.13 mN using the experimental calibration equation 13.

$${F}_{RMSE} = \sqrt{\frac{1}{n} {\sum }_{i = 1}^{n} {( {F}_{mfs , i} - {F}_{LC , i} )}^{2}} \qquad ( Eq . 14 )$$

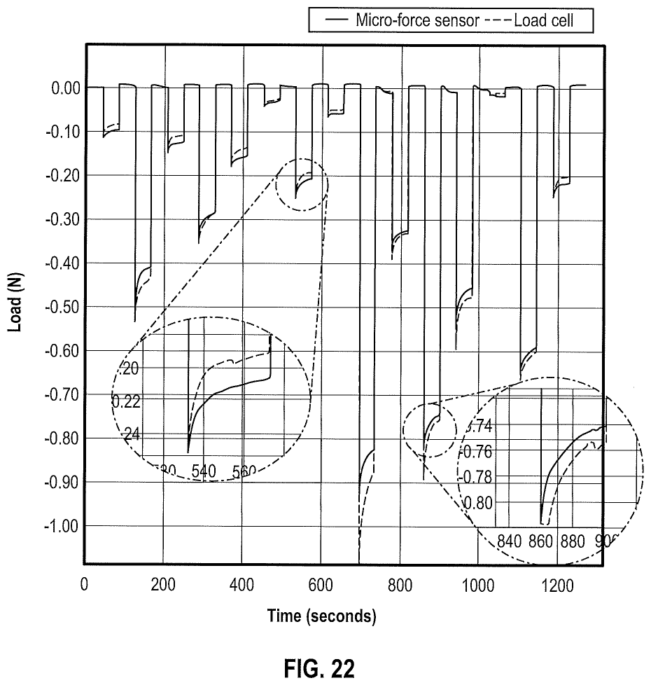

Accuracy. The accuracy of the sensor is evaluated as the deviation of a measured quantity from the load experienced by the load cell. The accuracy values to be evaluated depend on the specifications of the currently used equipment such as the micro-actuator position of ±2 μm and the load cell of 9.80 mN. FIG. 22 shows raw data collected from the load cell and the post-processed data from the strain gauge attached micro-force sensor for a single experiment. Similar behavior was observed for the other two experiments. The accuracy of the micro-force sensor is evaluated as the force root mean square error, F_{RMSE}, according to equation 14, where, F_{mfs} is the micro-force sensor load calculated by rearranging the calibration equation 13, F_{LC} is the actual load measured from the load cell i is the experiment run number, and n is the total number of experiments performed.

The analysis of the three measurement sets yields a F_{RMSE}=±28.6 mN (˜±2.9 grams) which is smaller than an accuracy of ±30 mN.

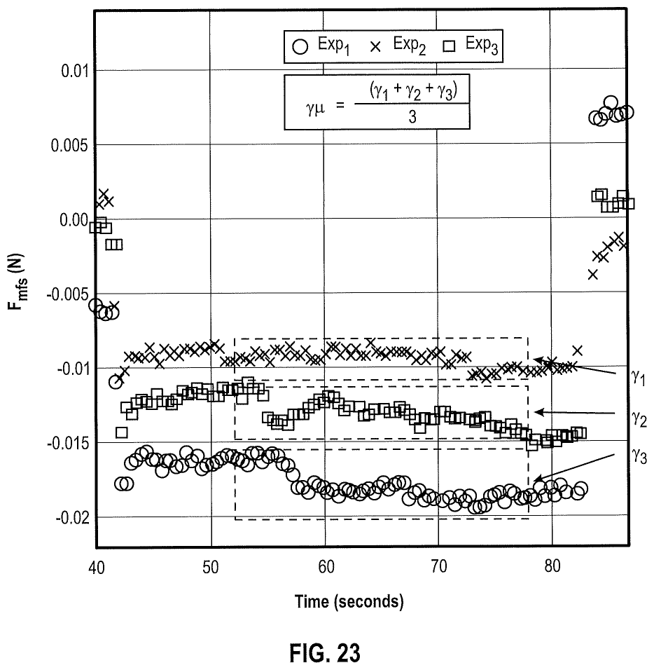

Precision. Precision refers to how closely individual measurements are in agreement with each other over a particular loading condition and is evaluated according to equation 15.

$$Precision = ( 1 - \vert \frac{{γ}_{i}}{{γ}_{μ}} \vert ) \times 100 \% \qquad ( Eq . 15 )$$

where, γᵢ is the average steady state strain obtained over a particular loading state of the sensor for an individual experiment and γ_{μ} is the average measurement for the three sets of experiments for the same loading condition. FIG. 23 shows representative sensor data for three experiments with the same applied displacement. The precision values for all the controlled displacement experiments (21 to 315 μm) were determined using equation 15.

The strain gauge-based sensor consistently demonstrated a high precision (≥87.22%) for all applied displacements except the first two (21 and 42 μm). Note that each displacement corresponds to an equivalent applied force on the sensor; for example, 21 and 42 μm correspond to 0.01 and 0.03 N respectively. The low precision for these smaller displacements could be attributed to frictional energy losses between the sensor head legs and sensor base slot feature.

Repeatability. Repeatability pertains to the level of consistency exhibited for multiple measurements obtained from a sensor or measurement system when subjected to identical input conditions. The repeatability of the sensor, ρ, is evaluated using equation 16, where ΔF is the maximum deviation across all sets of experiments, and Fz is the range of force measurement.

$$ρ = \pm ( \frac{Δ F}{{F}_{s}} ) \times 100 \% \qquad ( Eq . 16 )$$

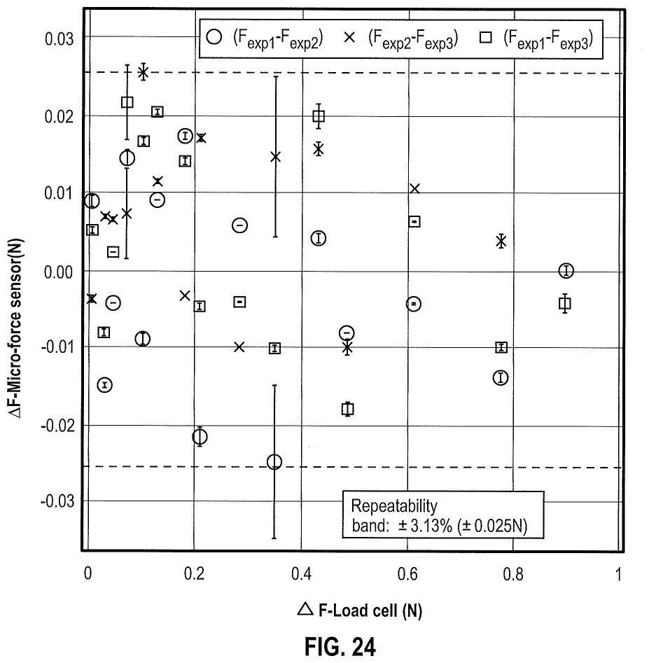

FIG. 24 presents the deviation in loads evaluated from measured strain gauge-based sensor data obtained between the three experiments. The repeatability band of the sensor was evaluated to be ±3.13% (˜+25 mN) corresponding to a strain change of 22.85μϵ.

Hysteresis. Hysteresis is the phenomenon where changes in the value of a physical attribute lag behind changes in the effect causing them. The largest deviation between the loading and unloading of the micro-force sensor over its defined operational range is used to assess hysteresis as a performance metric. The hysteresis is evaluated according to equation 17 where F_{loading} and F_{unloading} represent the calculated force during loading and unloading respectively.

$$ψ = Max \vert {F}_{loading} - {F}_{unloading} \vert \qquad ( Eq . 17 )$$

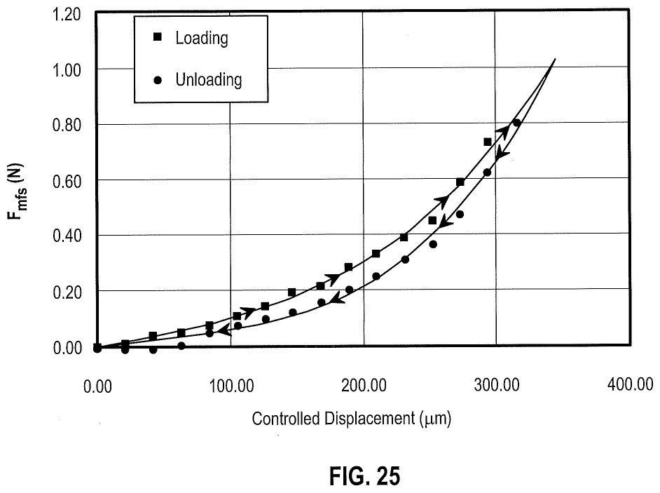

Hysteresis losses were estimated by subjecting the sensor to incremental loading intervals and allowing the sensor to reach steady state with each increment until the maximum displacement was reached. Then, the unloading profile followed a decrement from the maximum displacement at predefined intervals while allowing the sensor to reach steady state until the initial zero displacement. FIG. 25 shows the postprocessed hysteresis data from the micro-force sensor.

As the input displacement increases, the sensor response increases and provides a measurable output at the defined displacement. Using equation 8, a maximum hysteresis of 118 mN was calculated at a displacement of 273 μm. The hysteresis losses recorded from the sensor characterization experiments demonstrated that the assembled sensor release energy during the unloading phase. The hysteresis losses of the micro-force sensor could be attributed to multiple factors including the friction between the sensor head leg feature and the sensor base guide slots.

Performance Matrix Summary. The performance characteristics of the micro-force sensor based on the experimentally obtained and processed measurements are summarized in table 5. These results demonstrate the microforce sensor met the design specifications for its application in obtaining tissue relaxation responses during tissue palpation in confined spaces.

TABLE 5

| Characteristic | Specification |
|---|---|
| Diameter | 3.3 mm (sensor housing) |
| Force Range | 0-0.87N (normal force) |
| Sensitivity | 859.7 μϵ/N |
| Resolution | 0.13 mN |
| Accuracy | 28.6 mN |
| Precision | 87.22% |
| Repeatability band | ±3.13%(~±25 mN) |

### Additional Embodiments

Some additional, non-limiting, example embodiments are provided below.

Embodiment 1. A device for tissue evaluation, the device comprising: a sensor housing comprising: a sensor head configured to engage with a target tissue, and a sensor body or base, and a sensing element disposed within the sensor housing, wherein the device is configured to measure a reaction force of the target tissue when the sensor head is pressed and/or indented at a normal direction against the target tissue. In some aspects, the device is configured to measure a reaction force of the target tissue as a function of time.

Embodiment 2. The device of claim 1, wherein at least one of the sensor head and the sensor body are fabricated by an additive manufacturing process, such as a three-dimensional printing process. In some other embodiments the sensor head and/or body and/or sensing element are produced by an additive manufacturing process. In some other embodiments the sensor head and/or body and/or sensing element are produced by an injection molding process, an extrusion and thermoforming process, or any process not inconsistent with the technical objectives of the present technology.

Embodiment 3. The device of any of the preceding embodiments wherein the sensor housing has an outer diameter of less than or equal to about 3.5 mm.

Embodiment 4. The device of any of the preceding embodiments wherein the device is configured to operate under an applied force of up to about 1.2 N.

Embodiment 5. The device of any of the preceding claims wherein the device has a resolution of about 20 mN.

Embodiment 6. The device of any of the preceding claims, wherein the device has a safety factor of at least 3. In some further aspects, the device has a safety factor of at least 3.5.

Embodiment 7. The device of any of the preceding claims, wherein the sensor head has a hemispherical surface for engaging with the target tissue.

Embodiment 8. The device of any of the preceding claims, further comprising a strain gauge in operable communication with the sensing element.

Embodiment 9. The device of any of the preceding claims wherein the sensing element has a first end and a second end, wherein the first end is curved.

Embodiment 10. The device of any of the preceding claims, wherein the sensor head comprises a load transmitter configured to engage with the sensing element.

Embodiment 11. The device of any of the preceding claims, wherein the load transmitter is configured to contact the first end.

Embodiment 12. The device of any of the preceding claims, wherein the sensing element is fixedly or rigidly attached to the sensor body or base.

Embodiment 13. The device of any of the preceding claims, wherein the sensor head flexes the sensing element when the sensor head receives an applied force from the target tissue.

Embodiment 14. The device of any of the preceding embodiments, wherein the sensor head comprises at least two legs that engage with an inside cavity of the sensor body and are configured to allow free movement of the sensor head relative to the sensor body. In some aspects, the inside cavity is an inside guideway cavity.

Embodiment 15. The device of any of the preceding embodiments, wherein the device further comprises a biocompatible protective sheath or covering.

Embodiment 16. The device of any of the preceding embodiments, wherein the sensing element generates a signal corresponding to the reaction force of the target tissue.

Embodiment 17. A method for measuring a force and/or a reaction for of tissue comprising: inserting and/or implanting a sensor device according to claim 1 into a body such that the sensor head is substantially perpendicular to and in contact with a target tissue; receiving an applied force from the target tissue against the sensor head; causing the sensing element to deform in response to a displacement of the sensor head; and generating one or more signals corresponding to a reaction force of the target tissue.

Embodiment 18. The method of embodiment 17, wherein the one or more signals are generated by a strain gauge attached to the sensing element.

Embodiment 19. The method of embodiments 17 and/or 18, wherein the maximum accuracy error level of the sensor is about 3%.

Embodiment 20. The method of any of embodiments 17, 18, and 19, wherein the precision of the sensor is greater than 87%.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A device for tissue evaluation, the device comprising:
a sensor housing comprising:
a sensor head configured to engage with a target tissue, and
a sensor body; and
a sensing element disposed within the sensor housing, wherein the device is configured to measure a reaction force of the target tissue as a function of time when the sensor head is pressed against the target tissue.

2. The device of claim 1, wherein at least one of the sensor head and the sensor body are fabricated by an additive manufacturing process.

3. The device of claim 1, wherein the sensor housing has an outer diameter of less than or equal to about 3.5 mm.

4. The device of claim 1, wherein the device is configured to operate under an applied force of up to about 1.2 N.

5. The device of claim 1, wherein the device has a resolution of about 20 mN.

6. The device of claim 1, wherein the device has a safety factor of at least 3.5.

7. The device of claim 1, wherein the sensor head has a hemispherical surface for engaging with the target tissue.

8. The device of claim 1, further comprising a strain gauge in operable communication with the sensing element.

9. The device of claim 1, wherein the sensing element has a first end and a second end, wherein the first end is curved.

10. The device of claim 9, wherein the sensor head comprises a load transmitter configured to engage with the sensing element.

11. The device of claim 10, wherein the load transmitter is configured to contact the first end.

12. The device of claim 1, wherein the sensing element is fixedly attached to the sensor body.

13. The device of claim 11, wherein the sensor head flexes the sensing element when the sensor head receives an applied force from the target tissue.

14. The device of claim 1, wherein the sensor head comprises at least two legs that engage with an inside guideway cavity of the sensor body and are configured to allow free movement of the sensor head relative to the sensor body.

15. The device of claim 1, wherein the device further comprises a biocompatible protective sheath or covering.

16. The device of claim 1, wherein the sensing element generates a signal corresponding to the reaction force of the target tissue.

17. A method for measuring a reaction force of tissue, the method comprising:
inserting a sensor device according to claim 1 into a body such that the sensor head is substantially perpendicular to and in contact with a target tissue;
receiving an applied force from the target tissue against the sensor head;
causing the sensing element to deform in response to a displacement of the sensor head; and
generating one or more signals corresponding to a reaction force of the target tissue.

18. The method of claim 17, wherein the one or more signals are generated by a strain gauge attached to the sensing element.

19. The method of claim 17, wherein the maximum accuracy error level of the sensor is about 3%.

20. The method of claim 17, wherein the precision of the sensor is greater than 87%.
